(19) United States
(12) Patent Application Publication
(10) Pub. No.: US 2016/0194696 A1
(43) Pub. Date: Jul. 7, 2016

(54) Detecting, Sequencing and/or Mapping 5-Hydroxymethylcytosine and 5-Formylcytosine at Single-Base Resolution

(71) Applicant: NEW ENGLAND BIOLABS, INC., Ipswich, MA (US)

(72) Inventors: Shengxi Guan, Stoneham, MA (US); Zhenyu Zhu, Beverly, MA (US); Zhiyi Sun, Gloucester, MA (US); Nan Dai, Gloucester, MA (US)

(73) Assignee: New England Biolabs, Inc., Ipswich, MA (US)

(21) Appl. No.: 14/911,094

(22) PCT Filed: Aug. 7, 2014
(86) PCT No.: PCT/US2014/050157

§ 371 Date: Feb. 9, 2016

Related U.S. Application Data
(60) Provisional application No. 61/864,299, filed on Aug. 9, 2013.

(51) Int. Cl. C12Q 1/68

(52) U.S. Cl. CPC C12Q 1/6869; C12Q 1/6872

(57) ABSTRACT

This disclosure describes, among other things, compositions, kits and methods for identifying to 5-hydroxymethylcytosine (5hmC) in eukaryotic genomic DNA. The compositions and methods utilize a PvuRts1I family enzyme for digesting eukaryotic genomic DNA suspected of containing 5hmC or 5-formylcytosine (5fC) modified nucleotides providing an end of the DNA with a two base overhang suitable for ligating an adapter at a fixed distance 3′ from an 5hmC. Random fragmentation of the genomic DNA can generate a blunt end suitable for attaching a second adapter. The DNA can be sequenced and 5hmC and 5fC residues identified and located at a defined position in the eukaryotic genome. An enrichment step may be used that utilizes a chemoselective agent capable of being selectively added to DNA containing 5hmC. An example is a glucosyltransferase and a glucosyltransferase substrate comprising a chemo-selective group. This DNA can then be enriched by immobilization on a matrix that binds the chemoselective agent added to the DNA containing 5hmC and DNA that does not contain a chemo-selective group is removed by washing. Adapters can be added at one or both ends of the restriction endonuclease cleaved DNA after random fragmentation prior to or after an enrichment step.

### CROSS REFERENCE

This application is a §371 application of international application number PCT/US2014/050157 filed Aug. 7, 2014, which claims priority from U.S. provisional application No. 61/864,299 filed Aug. 9, 2013, herein incorporated by reference.

### GOVERNMENT RIGHTS

This invention was made with government support under GM096723 awarded by the National Institutes of Health. The government has certain rights in this invention.

### BACKGROUND

In mammals, 5-methylcytosine (5mC) plays important roles under physiological and pathological conditions (Klose, et al., Trends Biochem Sci, 31(2):89-97 (2006)). 5mC can be oxidized to 5-hydroxymethylcytosine (5hmC) by the ten-eleven translocation (TET) family of enzymes, including TET1, 2 and 3 (Kriaucionis, et al., Science, 324(5929):929-30 (2009); Tahiliani, et al., Science, 324(5929):930-5 (2009)). TET enzymes can further oxidize 5hmC to 5-formylcytosine (5fC) and 5-carboxylcytosine (5caC) successively (He, et al., Science, 333(6047):1303-7 (2011); Ito, et al., Science, 333(6047):1300-3 (2011)). The oxidation pathway from 5mC to 5fC/5caC followed by thymine-DNA glycosylase (TDG)-mediated base excision repair was proposed as a plausible mechanism for active DNA demethylation. The existence of three additional derivatives of 5mC (5hmC, 5fC and 5caC) in the mammalian genome adds another layer of complexity to DNA epigenetic dynamics. In the genome, 5hmC, 5fC and 5caC exist at very low abundances, causing technological challenges to study their distribution and biological functions.

In addition to serving as an intermediate during 5mC demethylation in DNA, 5hmC is also involved in various biological processes including embryonic stem cell (ESC) maintenance and differentiation (Williams, et al., EMBO Rep, 13(1):28-35 (2012); Branco, et al., Nat Rev Genet, 13(1):7-13 (2012); Wu, et al., Genes Dev, 25(23):2436-52 (2011); Koh, et al., Cell Stem Cell, 8(2):200-13 (2011)), normal hematopoiesis and malignancies (Ko, et al., Nature, 468(7325):839-43 (2010); Moran-Crusio, et al., Cancer Cell, 20(1):11-24 (2011); Quivoron, et al., Cancer Cell, 20(1):25-38 (2011), and zygote development (Iqbal, et al., Proc Natl Acad Sci USA, 108(9):3642-7 (2011); Wossidlo, et al., Nat Commun, 2:241 (2011); Gu, et al., Nature, 477(7366):606-10 (2011)). 5hmC is strongly depleted in human cancer cells compared with normal tissue (Haffner, et al., Oncotarget, 2(8):627-37 (2011); Lian, et al., Cell, 150(6):1135-46 (2012); Kudo, et al., Cancer Sci, 103(4):670-6 (2012); Jin, et al., Cancer Res, 71(24):7360-5 (2011)). However, whether the phenomenon contributes to tumor progression is still unknown. Due to the extremely low level of 5fC/5caC, it remains unclear whether these two cytosine variants have other regulatory functions besides serving as an intermediate of DNA 5mC demethylation.

Genome-wide profiling methods rely on affinity between 5hmC/5fC or its derivatives and antibody/chemicals (Ficz, et al., Nature, 473(7347):398-402 (2011); Wu, et al., Genes Dev, 25(7):679-84 (2011); Pastor, et al., Nature, 473(7347):394-7 (2011); Song, et al., Nat Biotechnol, 29(1):68-72 (2011); Shen, et al., Cell, 153(3):692-706 (2013); Song, et al., Cell, 153(3):678-91 (2013)). Antibody-based profiling methods can be biased to heavily modified regions (Pastor, et al. (2011)). To avoid this bias, a selective chemical labeling (Seal)-based method was developed and applied on both 5hmC and 5fC genome-wide profiling (hC-Seal and fC-Seal) (Song, et al., (2011); Song, et al., Cell, 153:678-691 (2013)) using T4 β-glucosyltransferase (T4-BGT) to add an azide-modified glucose moiety to 5hmC on the DNA. A biotin group can then covalently link to the azide group via copper-free click chemistry coupling permitting selectively pull-down by streptavidin beads.

The genome-wide profiling methods described above lack precise sites of 5hmC/5fC and cannot reveal the relative abundance of each modification site.

OxBS-seq and TAB-seq (Booth, et al., Science, 336(6083):934-7 (2012); Yu, et al., Cell, 149(6):1368-80 (2012)) are 5hmC single-base resolution mapping technologies that utilize bisulfite conversion. These methods have several disadvantages. For example during bisulfite treatment DNA can be degraded, bias can be introduced during PCR amplification (Grunau, et al., Nucleic Acids Res, 29(13):E65-5 (2001)), and sequencing depth for each cytosine must be high in order to detect low-levels of 5hmC (Yu, et al., (2012)).

In bisulfite sequencing, C, 5fC and 5caC are read as T; while 5mC, 5hmC and beta-glucosyl-5-hydroxymethylcytosine (5gmC) are read as C. In the oxBS-seq method, 5hmC is selectively oxidized to 5fC by potassium perruthenate (KRuO4) to achieve different 5hmC readout with or without oxidation (Booth, et al., (2012)). However, during the oxidation reaction, DNA damage and degradation is induced. In the TAB-seq method, 5hmC is first glucosylated to 5gmC and then all genomic 5mC is converted to 5caC by TET1, so that only 5hmC is intact while all other cytosine derivatives are deaminated by bisulfite (Yu, et al., (2012)). If 95% of 5mC is ideally converted to 5caC, the remaining 5% of 5mC still exists in the final 5hmC library. Among all tissues, brain contains the highest level of 5hmC (Ito, et al., (2011)). If the molar ratio between 5mC and 5hmC is 5:1 in the brain, 20% of the final 5hmC library contains 5mC contaminants.

Sensitive, non-biased 5hmC/5fC single-base-resolution sequencing method and genome mapping methods would greatly facilitate the diagnostic dividend of determining when and where 5hmC occurs in the genome.

### SUMMARY

In general, in one aspect, a method is provided for sample analysis, that includes digesting eukaryotic genomic DNA comprising 5hmC using a PvuRts1I-family restriction endonuclease to form a DNA having a first end, wherein the first end has a single strand overhang for example, a 3′ two random base overhang on a strand of the DNA having a 5hmC. The eukaryotic genomic DNA may be randomly fragmented for example to a size of less than 500 bases (i) prior to restriction endonuclease digestion, or (ii) after restriction endonuclease digestion. Random fragmentation may be achieved enzymatically or by sonication, shearing or nebulization. An adapter may be ligated to the first end; and the presence and the position of 5hmC in the eukaryotic genomic DNA detected by sequencing the adaptor ligated DNA.

In one aspect, the method includes selectively adding a chemoselective group to the 5hmC prior to sequencing the adapter ligated DNA. The chemoselective group may be added at a reaction temperature of at least 37° C. enzymatically, for example, using a glucosyltransferase and a glucosyltransferase substrate; or by other means.

In one aspect, the chemoselective group on the DNA may be reacted with a capture molecule that comprises an affinity moiety and optionally a cleavable linker such as a disulfide bond. The DNA may be reversibly captured via the affinity moiety such as biotin on a matrix and released from the matrix and released by cleaving the cleavable linker by for example, reducing the disulfide bond.

In one aspect, the PvuRts1I-family restriction endonuclease, the glucosyltransferase, the glucosyltransferase substrate and the genomic DNA may be combined in a single reaction vessel.

In another aspect restriction endonuclease activity may be removed prior to ligating the adapter for example, either by temperature inactivation of the enzyme or by removal of the enzyme by column chromatography.

In another aspect, an amount of the restriction endonuclease may correspond to a molar ratio of the restriction endonuclease to total 5hmC in the eukaryotic DNA of at least 0.5:1.

In another aspect, a second adapter may be added to a second end for amplifying the DNA between the adapters at the first end and the second end.

In another aspect, a cytosine in a genomic DNA treated as described above may be annotated as being a 5hmC or 5fC in the eukaryotic genomic DNA according to its location 11-12 nucleotides from the first end of the DNA.

In one aspect, genomic DNA may be treated with NaBH₄ prior to restriction endonuclease digestion.

In one aspect, a kit containing a PvuRts1I-family restriction endonuclease, a glucosyltransferase and a glucosyltransferase substrate that comprises a chemo-selective group and a buffer and instructions for use at an initial temperature of room temperature (RT) followed by an incubation at least 37° C. is provided.

In general, in one aspect, a preparation is provided that includes a PvuRts1I-family restriction endonuclease and a eukaryotic DNA wherein the molar ratio of the restriction endonuclease to 5hmC in eukaryotic DNA is at least 0.5:1. The preparation may further include a glucosyltransferase and a glucosyltransferase substrate that comprises a chemo-selective group. The preparation may additionally include an adapter having at least a two nucleotide 3′ overhang of random sequence and a 5′ phosphate.

### DESCRIPTION OF FIGURES

The skilled artisan will understand that the drawings, described below, are for illustration purposes only. The drawings are not intended to limit the scope of the present teachings in any way.

FIGS. 1A and 1B shows PvuRts1I specificity for 5hmC DNA using a double stranded DNA fragment of 54 base pairs having a 5hmC at position 20 on one strand to generate 32 bp and 22 bp cleavage products.

FIG. 1A shows the cleavage pattern when PvuRts1I which cuts double stranded DNA at a fixed distance from the 5hmC regardless of the nucleotide sequence downstream.

FIG. 1B shows the cleavage pattern for DNA digested with PvuRts1I and analyzed by gel electrophoresis. Different substrates were digested with 10-fold serial diluted PvuRts1I. The two bands indicated by arrows correspond to the 22 bp and 32 bp fragments. Lane 1 in each dilution series was the preferred concentration of PvuRts1I irrespective of downstream nucleotide sequences.

FIG. 2 shows a schematic illustration of Pvu-Seal-Seq.

Three different fragments of DNA containing either a 5hmC, 5mC or C are reacted with PvuRts1I (1) and each fragment is completely (5hmC) or partially cleaved (5mC and 5fC) where the cleaved fragments are characterized by a 2 nucleotide 3′ single strand overhang. The cleaved DNA is reacted with T4-BGT+UDP-6N3-Glc (2). The T4-BGT converts 5hmC to 6N3-gmC but does not react with 5mC or C. An adapter (P1) is then ligated to the single strand overhang on each of the three types of cleavage product (3). The DNA is then reacted with DBCO-PEG3-S-S-Biotin using Click chemistry (Click Chemistry Tools, Scottsdale, Ariz.) which connects azide group with Biotin (4). The biotin labeled DNA is pulled down by streptavidin coated beads (5). The 5mC and C containing DNA fragments are removed by washing. A second adapter (P2) is ligated to the other end of the Biotin labeled DNA (6) and then the DNA fragments containing the modified cytosine are released in the presence of DTT (7). The resulting DNA fragment carrying an adapter at each end can be sequenced using next generation sequencing techniques (8).

FIG. 3A-3D show an analysis of the sensitivity of embodiments of the method for detecting 5hmC regardless of sequence context in E14 genomic DNA.

FIG. 3A shows the results of a genome-wide map of 5hmC sites at single-base resolution in the mouse embryonic stem cells. Genomic DNA from mouse E14 cells was used to generate two replicate 5hmC libraries. The weblogo shows the frequency of each nucleoside at each position (Crooks, et al., Genome Research, 14:1188-1190 (2004)).

FIG. 3B shows a pie chart in which 76% of the 5hmC sites were in CpG context and the remaining 24% of 5hmC sites were in CH context (17% in CHH and 7% in CHG, where H=A, C or T) for two overlapping libraries.

FIG. 3C shows that the overlapping ratio of 5hmCG sites (82%) was much higher than that of the 5hmCH sites (38%).

FIG. 3D shows that the average copy number of 5hmCpG sites is significantly higher than that of the 5hmCH sites for both overlapping sites and non-overlapping sites.

FIG. 4A provides a comparison of modified cytosines at CpG sites and non-CpG sites using Pvu-Pull down-Seq and TAB-Seq. Pvu-Pull down-Seq for a single library detected 33.8% 5hmC/ATC (24.9×10⁶ 5 hmC sites) compared with TAB-seq on the same samples which detected only 1.3% 5hmC/ATC sequences (2×10⁶ 5 hmC sequences). This demonstrated that Pvu-Pull down-Seq is at least 10-20 fold more sensitive than TAB-seq for 5hmC detection.

FIG. 4B provides a comparison of Pvu-Pull down-Seq and TAB-Seq showing that bias could not be detected. Pvu-Pull down-Seq detected about 25% 5hmC/ATG which is the same as was detected using TAB-Seq where TAB-Seq has been previously shown not to have bias with respect to downstream nucleotides. Because the results were similar for 5hmC using Pvu-Pull down-Seq and TAB-Seq, it could be concluded that Pvu-Pull down-Seq does not have any downstream sequence bias.

FIG. 5 provides a cartoon of an embodiment of a method for analyzing genomic DNA using Pvu-Pull down-seq with T4-BGT and UDP-Glc. 5hmC residues were converted to 5gmC, which prevented 5hmC from being pulled down in the later procedures. NaBH₄ was then used to reduce 5fC to 5hmC, followed by the Pvu-Pull down-Seq procedure.

FIG. 6 shows the results of reducing 5fC to 5hmC by NaBH4. A 1.6 kb PCR products with all Cs replaced by 5fC was incubated with 100 mM NaBH₄ at RT for 1 hour. The product was broken down into single nucleosides and was subjected to LC/MS analyses. 5fC was converted to 5hmC with an efficiency close to 100% enabling the study of distribution of 5fC.

FIG. 7A-7C show distributions of 5fC sites in two 5fC libraries from the same batch of E14 genomic DNA used for 5hmC library constructions.

FIG. 7A shows that 75% of overlapping 5fC sites were in a CpG context and 25% were in a CH context (17% is in CHH and 8% is in CHG). Similar to 5hmC (see FIG. 3B), overlapping 5fC sites had significantly higher average copy number (8.6) than non-overlapping sites (4.7) (Student's T test, P-value <1.0E-6).

FIG. 7B shows that the 5fCpG sites had significantly higher average copy number (7.1) than the 5fCH sites (4.8), and the overlapping ratio of 5fC in CpG context (55%) was significantly higher than that in CH context (20%) (FIG. 7B).

FIG. 7C shows that the 5fC regional density in 100 bp sliding windows across the entire genome revealed a good correlation between the two libraries, especially at regions with high 5fC level (Pearson correlation=0.99; Spearman rank correlation=0.51). This demonstrates that individual 5fC sites are transient and dynamic in contrast to hotspot regions for 5fC distributions which appear to be relatively stable at a given developmental stage.

FIG. 8A-8B show 5hmC and 5fC distributions in genic regions.

FIG. 8A shows that globally, 5hmCpG and 5fCpG sites had similar distributions in genic regions. After normalizing to the background CpG density, both 5hmCpG and 5fCpG densities dropped near transcription start sites (TSS) and remained low at the 5′UTR, but not at the 3′UTR. There was very little difference in the normalized modification levels between exons and introns. Within exon and intron regions, both 5hmCpG and 5fCpG appeared to gradually increase from the 5′ end to the 3′ end. The 5fCpG distribution also resembled the distribution of its precursor 5hmCpG.

FIG. 8B show that 5hmCH and 5fCH had distinct profiles in genic regions compared with 5hmCpG and 5fCpG. Normalized 5hmCH and 5fCH levels were elevated in coding regions in comparison to non-coding regions. In contrast to 5hmCpG and 5fCpG profiles, 5hmCH and 5fCH were not depleted near TSS. In addition, 5hmCH and 5fCH gradually decreased towards TTS.

FIG. 9A-9F show 5hmC and 5fC distributions at specific identified protein-DNA binding sites. The occurrence of a specified nucleotide is mapped at a particular genomic location where

FIG. 9A shows the prevalence of specific modified nucleotides in the TET1 binding site sequence,

FIG. 9B shows the prevalence of specific modified nucleotides in the CTCF binding site sequence,

FIG. 9C shows the prevalence of specific modified nucleotides in the P300 binding site sequence,

FIG. 9D shows the prevalence of specific modified nucleotides in the Nanog binding site sequence,

FIG. 9E shows the prevalence of specific modified nucleotides in the Tcfcp2I1 binding sequence and

FIG. 9F shows the prevalence of specific modified nucleotides in the Stat 3 binding site sequence.

FIG. 10A-10D show correlations between histone modification marks and the distribution of 5hmC and 5fC.

FIG. 10A shows that both 5hmC and 5fC were depleted at H3K4me3 chromatin modification sites.

FIG. 10B shows that 5hmC and 5fC were enriched at repressive chromatin loci marked by H3K27me3).

FIG. 10C shows that 5hmCs and 5fCs were enriched at active enhancers (H3K4me1 with H3K27Ac).

FIG. 10D shows that 5hmCs and 5fCs were enriched at poised (H3K4me1 without H3K27Ac) enhancers where enrichment was greater than in FIG. 10C showing a close correlation between DNA modification and transcription regulation.

### DEFINITIONS

Before describing exemplary embodiments in greater detail, the following definitions are set forth to illustrate and define the meaning and scope of the terms used in the description.

As used herein, the term “glucosyltransferase” refers to an enzyme that catalyzes the transfer of a B-D-glucosyl residue from UDP-glucose to a hydroxymethylcytosine residue in DNA. T4-BGT (Tomaschewski, et al, Nucleic Acids Res., 13: 7551-7568 (1984)) is an example of a glucosyltransferase.

As used herein, the term “glucosyltransferase substrate that comprises a chemo-selective group” includes, for example, a UDP-Glc derivative that contains a chemo-selective group that can be transferred to a DNA substrate using a glucosyltransferase. Examples of such substrates are described in, e.g., Dai, et al., Chembiochem, 14: 2144-2152 (2013) and Song, et al, (2011), which are incorporated by reference herein. This term includes substrates that contain 6-N3-glucose, as well functional equivalents thereof (e.g., substrates that contain non-azide chemo-selective groups and substrates that contain glucosamine).

The term “chemoselective group” refers to a reactive group that is not already present in the sample under study, i.e., an “orthogonal” group. For example, a thiol group (which is reactive with iodoacetamide) is orthologous if the sample does not contain any thiol groups. Likewise, the reactive groups used in click chemistry (e.g., azide and alkyne groups) can be used. Chemoselective functional groups of interest include, but are not limited to, thiol, amide, aldehyde, thiophosphate, iodoacetyl groups, maleimide, azido, alkynyl (e.g., a cyclooctyne group), phosphine groups, amide, click chemistry groups, groups for staudinger ligation, and the like.

The term “capture molecule” refers to a molecule that can be used to capture compounds that have a chemoselective group. Capture molecules are bifunctional in that they contain a group that covalently reacts with a chemoselective functional group (e.g., an active ester such as an amino-reactive NHS ester, a thiol-reactive maleimide or iodoacetamide groups, an azide group or an alkyne group, etc.), and a purification tag (referred to herein as the “affinity moiety”), such as a biotin moiety, that can be used to anchor compounds containing the tag to a substrate, e.g., beads or the like.

As used herein, the term “biotin moiety” refers to an affinity agent that includes biotin or a biotin analogue such as desthiobiotin, oxybiotin, 2′-iminobiotin, diaminobiotin, biotin sulfoxide, biocytin, etc. biotin moieties bind to streptavidin with an affinity of at least 10⁻⁸M. A biotin affinity agent may also include a linker, e.g., -LC-biotin, -LC-LC-biotin, -SLC-biotin or -PEGn-biotin where n is 3-12.

As used herein, the term “cleavably linked” refers to a linkage that is selectively breakable using a stimulus (e.g., a physical, chemical or enzymatic stimulus) that leaves the moieties to which the linkages joins intact. Several cleavable linkages have been described in the literature (e.g., Brown, Contemporary Organic Synthesis, 4(3); 216-237 (2007)) and Guillier, et al., Chem. Rev., 1000:2091-2157 (2000)). A disulfide bond (which can be broken by DTT) and a photo-cleavable linker are examples of cleavable linkages.

As used herein, the term “identifiable location” refers to a position in a fragment that is known before the fragment is sequenced. For example, in some cases, one may know that there is a modified cytosine at 11 or 12 nucleotides from the end of a fragment (i.e., from site of cleavage site), without knowing the sequence of the fragment.

As used herein, the term “overhang of random sequence” refers to a population of overhangs that are composed of Ns, where N can be any nucleotide. For example, a two base overhang of random sequence has an overhang of sequence NN, where N can be any nucleotide. In this example, the individual overhangs are of sequence N₁N₂, where N₁ and N₂ are independently G, A, T or C.

As used herein, the term “random fragmentation” or “random cleavage” refers to fragmentation or cleavage achieved using a nonspecific nuclease or physical methods such as shearing by sonication.

As used herein, the term “PvuRts1I-family restriction endonuclease” refers to the family of restriction endonucleases described in Wang, et al., Nuc. Acids. Res., 39: 9294-9305 (2011). PvuRts1I, PpeHI, BbiDI, AbaSDFI, YkrI, PatTI, SpeAI, BmeDI, EsaNI are examples of PvuRts1I-family restriction endonucleases. Further PvuRts1I-family restriction endonucleases and variants thereof are described in US Patent Publication No. 2015/0004596. Where the term “PvuRts1I” is used, it should be understood that this encompasses variants with at least 80% or 85% or 90% or 92% or 95% or 97% or 98% or 99% amino acid sequence identity. Where the term “PvuRts1I-family restriction endonuclease” is used, this term is intended to include enzymes have at least 80% or 85% or 90% or 92% or 95% or 97% or 98% or 99% sequence identity to the identified members of the family.

Unless indicated to the contrary, reference to a particular enzyme (e.g., PvuRts1I, AbaSI, MspI, a PvuRts1I-family restriction endonuclease, etc.) is intended to encompass the wild type enzyme as well as variants of the wild type enzyme that are functional and have an amino acid sequence that is at least 80% or 90% or 95% identical to the wild type enzyme, and fusions thereof.

The molar amount of 5hmC in a eukaryotic genome can be calculated based on the data presently available namely that about 20% of all bases in a genome are cytosine of which a small percentage are 5hmC (see for example, Ito, et al., (2011)). Accordingly, the percentage of 5hmC in the genome can vary from tissue to tissue and, in some embodiments, the percentage of 5hmC in a genome may vary from about 0.001% to 0.2%. For example, in the human genome, the percentage of 5hmC in is about 0.6%-0.7% of total cytosine in the brain (i.e., about 0.1% of all nucleotides), about 0.1% of total cytosine in embryo tissue (i.e., about 0.02% of total nucleotides), and about 0.03% of all cytosine in the thymus (i.e., about 0.002% of all nucleotides). The approximate molarity can be calculated from the numbers in the range provided. In some embodiments of the method, it is assumed that the genome contains 0.1% 5hmC/Cytosine (applicable to kidney, lung, pancreas, liver), 0.6% 5hmC/Cytosine (for brain tissue), and 0.03% 5hmC/Cytosine for spleen, thymus and embryonic cells.

### DETAILED DESCRIPTION

Before various embodiments are described in greater detail, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the present disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, the some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

Before describing various embodiments in more detail, it is noted that the method may be implemented using any restriction endonuclease that can cleave hydroxymethylated DNA. For example, in order to implement this method using an enzyme other than a PvuRTS1I-family member, the sequence of the overhang of the adaptor may be changed so that it is complementary to the predicted cleavage site. MspI and other members of the XXYZ family, which can all cleave hydroxymethylated DNA, are examples of such enzymes.

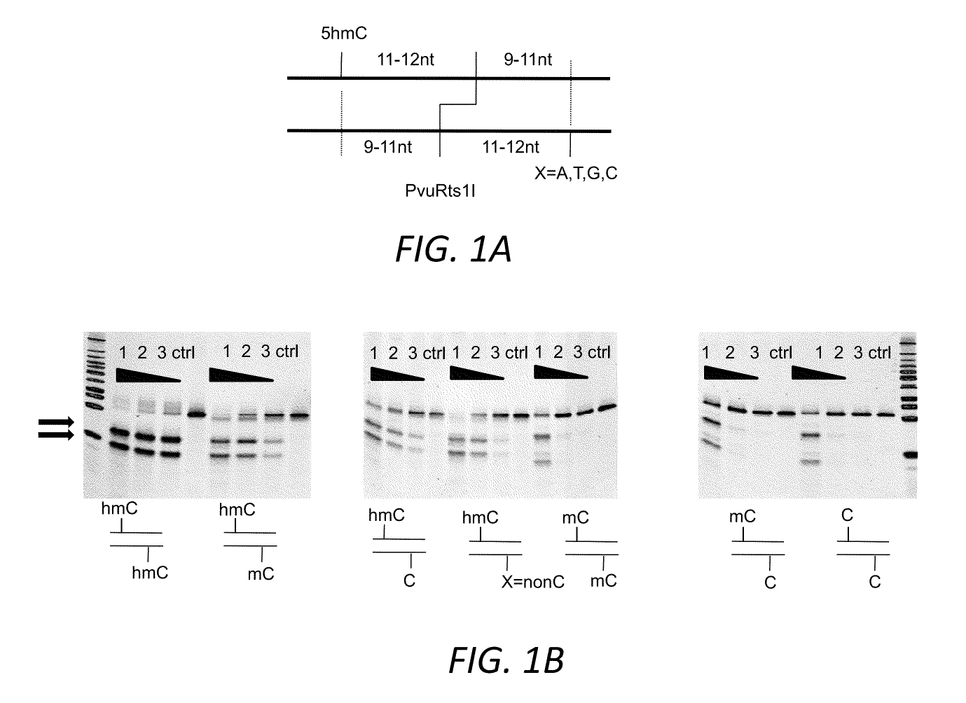

Some embodiments of the method rely on a PvuRts1I-family restriction endonuclease, which cuts to produce a two base overhang of random sequence at a fixed distance downstream from a 5hmC. For example, if the top strand contains a 5hmC, then PvuRts1I cuts the top strand at a site that is either 11 nucleotides or 12 nucleotides 3′ to the 5hmC, and the bottom strand at a site that is 9-11 bases 3′ to the 5hmC, at the sequence ^{hm}CN₁₁₋₁₂/N₉₋₁₀G (FIG. 1A). In addition to digestion using a PvuRts1I-family restriction endonuclease the sample should also be randomly fragmented so that a substantial portion of the fragments contains only one end with a two base overhang of random sequence.

5hmCs can be modified by a chemoselective group using for example, a DNA glucosyltransferase (e.g., BGT). In one embodiment, the chemoselective group can be linked to a capture molecule e.g., biotin using for example, click chemistry where the chemoselective moiety may be an azido or alkynyl group. The chemoselective group can then bind DNA containing 5hmC to a matrix (e.g., straptividin beads or the like) via the capture molecule to achieve enrichment of the bound DNA. In some embodiments, the capture molecule may contain a cleavable linker. In these embodiments, the cleavable linker may be cleaved to release the DNA from the matrix.

In some embodiments, the digestion and glucosyltransferase treatment steps can occur in a single vessel with no addition reagents being added during the course of the reaction. For example, in some cases, the digestion may be done at approximately room temperature (e.g., at a temperature of 20° C.-25° C.) and the glucosyltransferase treatment step may be done at a temperature of at least 37° C. for example, at 37° C.

A first double stranded adaptor (containing two nucleotide 3′ overhang of random sequence) can be ligated to the two base overhang at any point in the method after cleavage with a PvuRts1I family enzyme. Random fragmentation of the eukaryotic genome can be performed before or after digestion of DNA with a PvuRrtsll family enzyme at any stage in the method preferably prior to an enrichment step for DNA containing 5hmC. A second adaptor can be ligated by any suitable method to the other end of the DNA which may be partially or completely blunt ended where this ligation can be performed at any stage in the method but preferably after random fragmentation of the eukaryotic genome. The enriched DNA can be amplified using primers that hybridize to the adaptor sequences, and sequenced. The hydroxymethylated nucleotide can be identified immediately because it is a defined distance from the end of the enriched DNA. Specifically, if the top strand is sequenced, then the cytosine that is 11 or 12 bases from the 3′ end of the DNA corresponds to a 5hmC in the genome. Likewise, if the bottom strand is sequenced, then the guanine that is 9 or 10 bases from the 5′ end of the fragment base pairs with a 5hmC in the genome. Because the hydroxymethylated nucleotides can be immediately identified by their position in each of the sequences, the method facilities genome annotation in an automated manner (i.e., by a computer) using raw or processed sequence.

An enriching step of the method separates the hydroxymethylated DNA from non-hydroxymethylated DNA, which removes: a) fragments resulting from star activity of the PvuRts1I-family restriction endonuclease (e.g., which might result in cleavage downstream from a 5mC or cytosine instead of from a 5hmC) and b) fragments that are adjacent to the hydroxymethylated fragments, i.e., fragments that are on the “other side” of the cleavage site that have the same two base 3′ overhang but do not contain a 5hmC.

The random (non-PvuRts1I) fragmentation step (which may be done by any suitable method, e.g., nonspecific nuclease, shearing or the like) should be done before enrichment of the hydroxymethylated DNA so that, after the fragments are sequenced, there is no confusion about which end of a fragment contains the 5hmC. Specifically, without a random fragmentation step, both ends of every DNA in the sample after PvuRts1I restriction endonuclease digestion should contain a 3′ overhang of two random nucleotides (NN). Fragmenting the sample prior to enrichment of the hydroxymethylated fragments: a) ensures that all of the DNA contains a 5hmC close to the end that contains the 3′ overhang, and b) allows the sequence containing the 5hmC to be read from both directions, if desired.

Embodiments of the methods and compositions provide but is not limited to a means to achieve one or more of the following: a sensitive method for detection of 5hmC and 5fC with single base resolution at a genome wide scale; detection of rare occurrences of 5hmC and 5fC within a CpG context or in a non-CpG context; correlation of the occurrence of 5hmC and 5fC in genomic sites associated with transcriptional regulation such as transcription factor binding sites, enhancer sequences and other regulator protein-DNA binding sites; correlation of the distribution of 5hmC and 5fC with the occurrence and progression of different types of cancer cells and tissues and other pathological conditions; and correlation of the distribution of 5hmC and 5fC with one or more conditions associated with normal or abnormal development, health, resistance or sensitivity to infectious agents, aging or lack of aging or other phenotype.

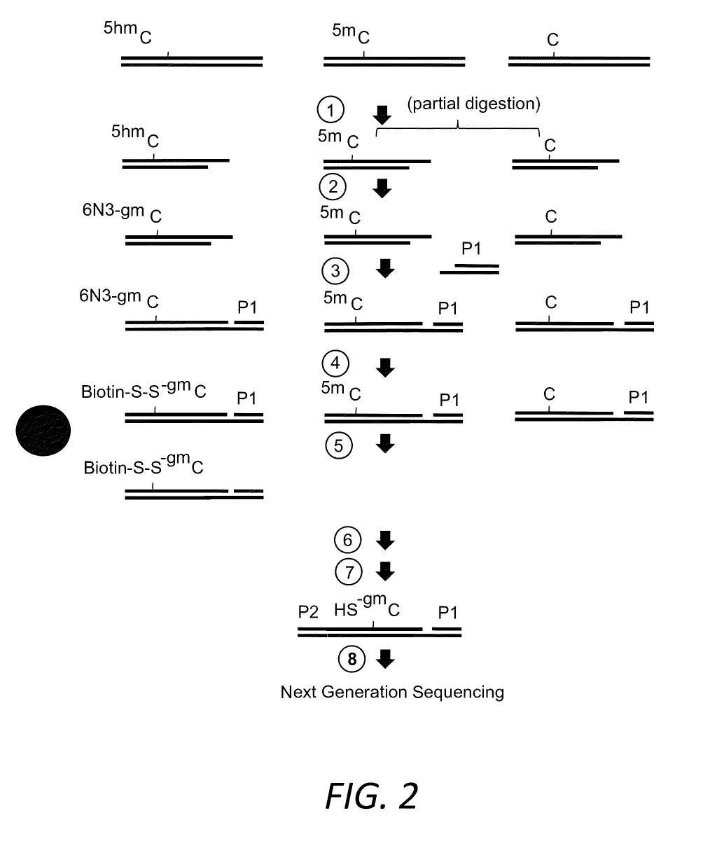

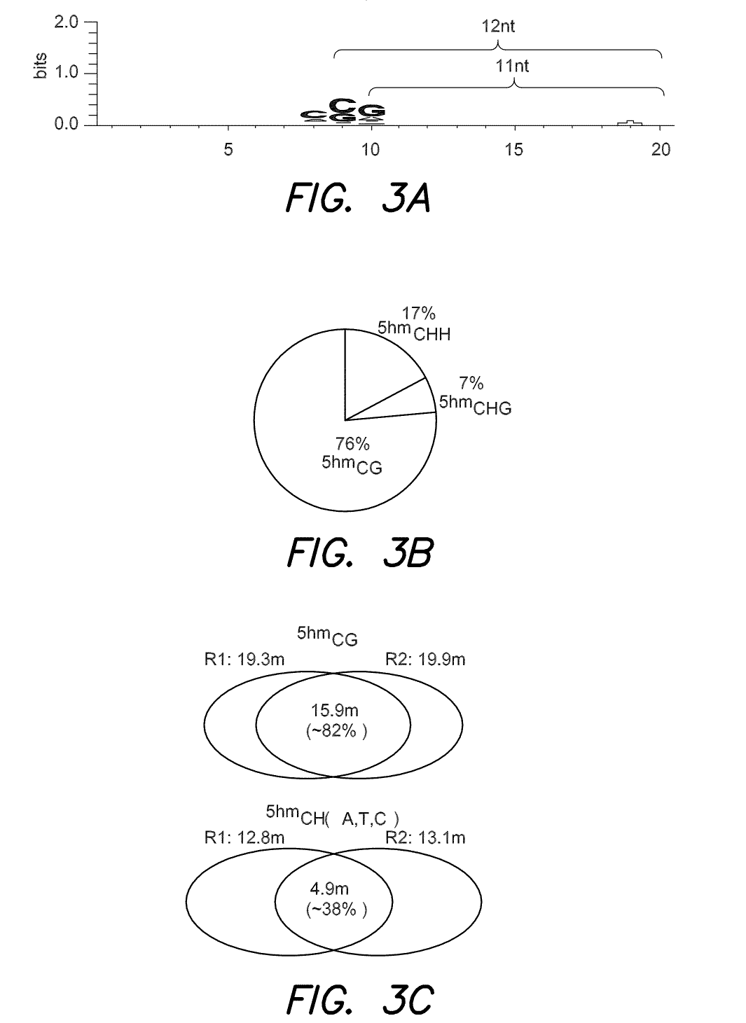

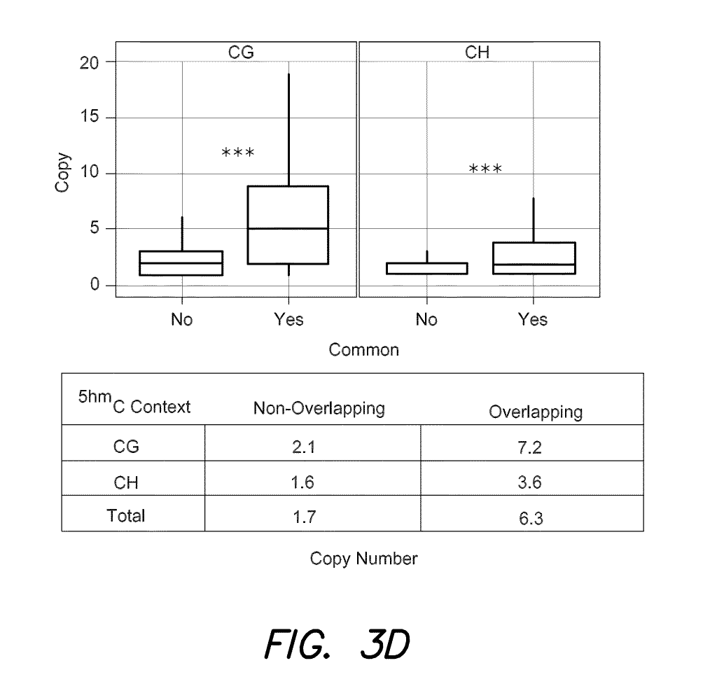

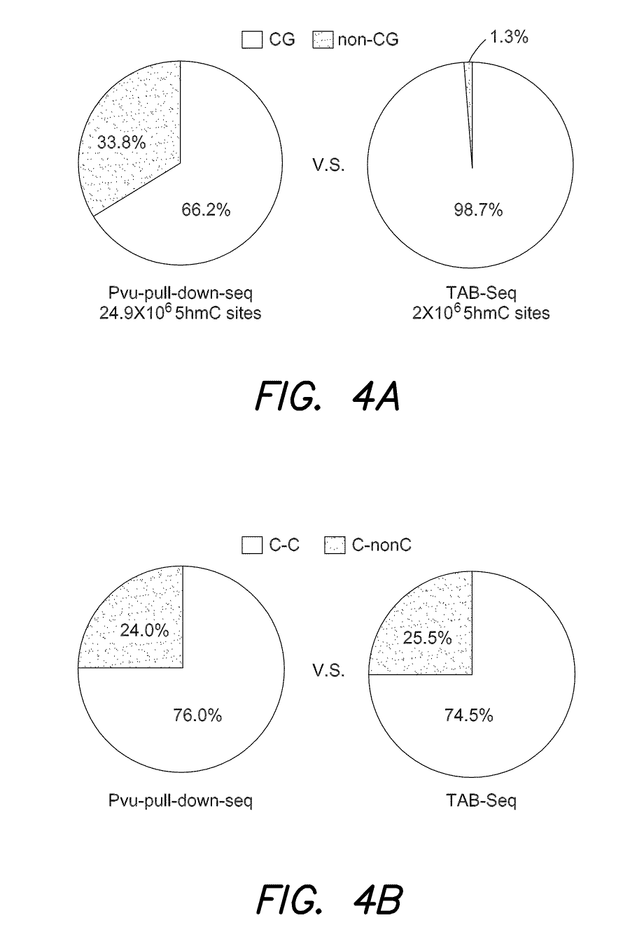

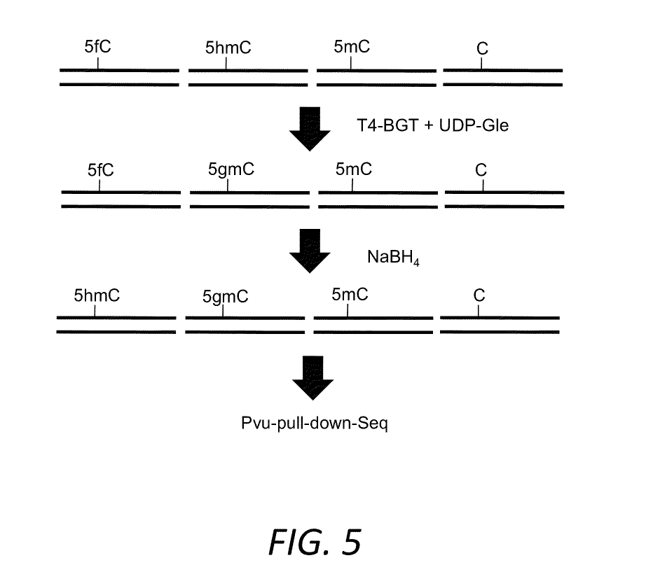

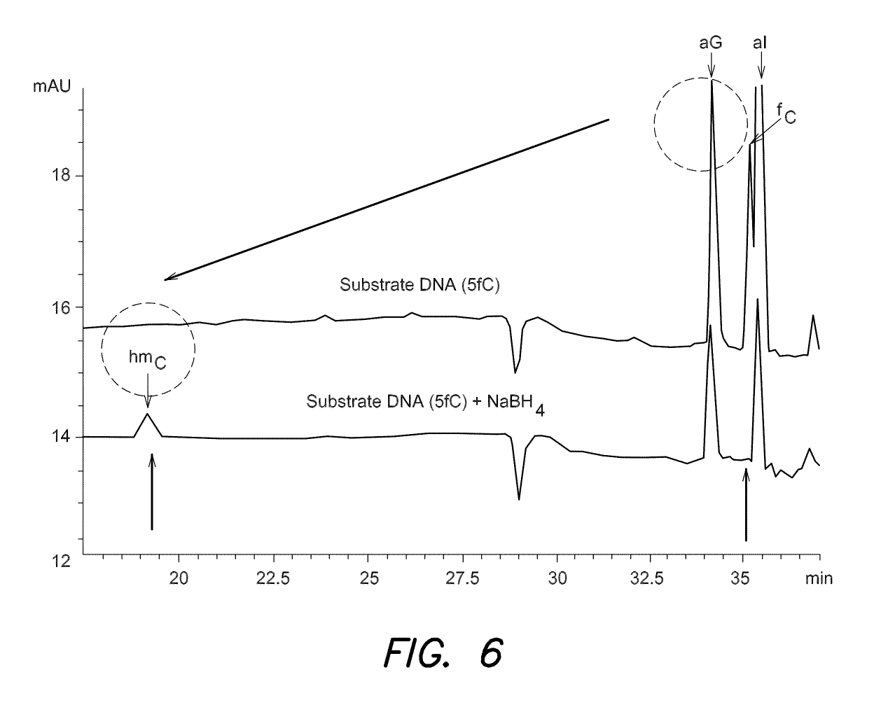

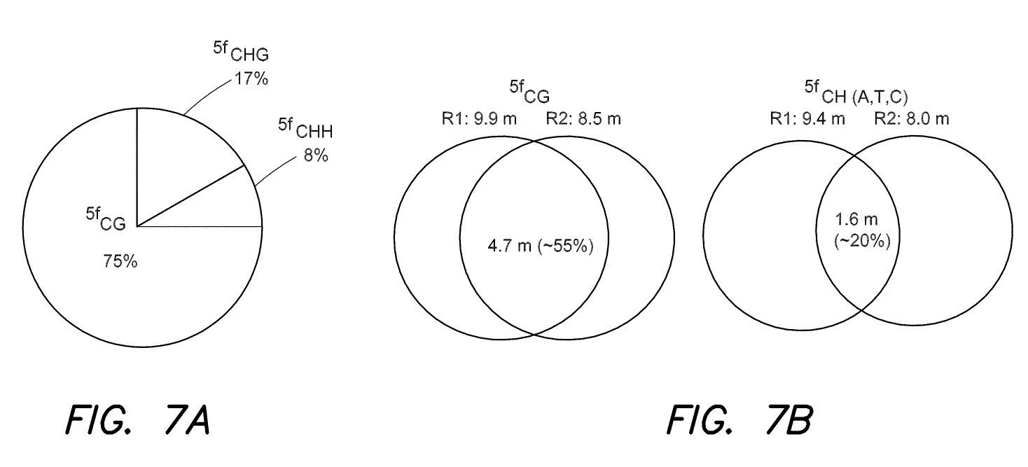

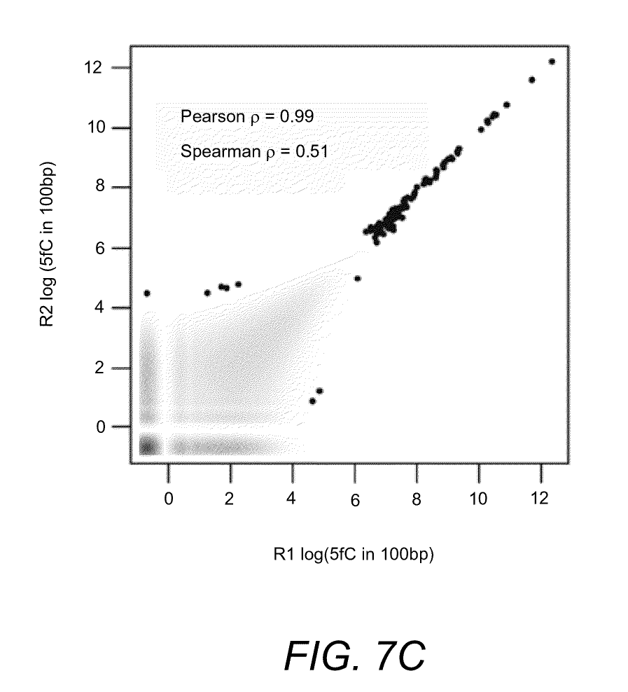

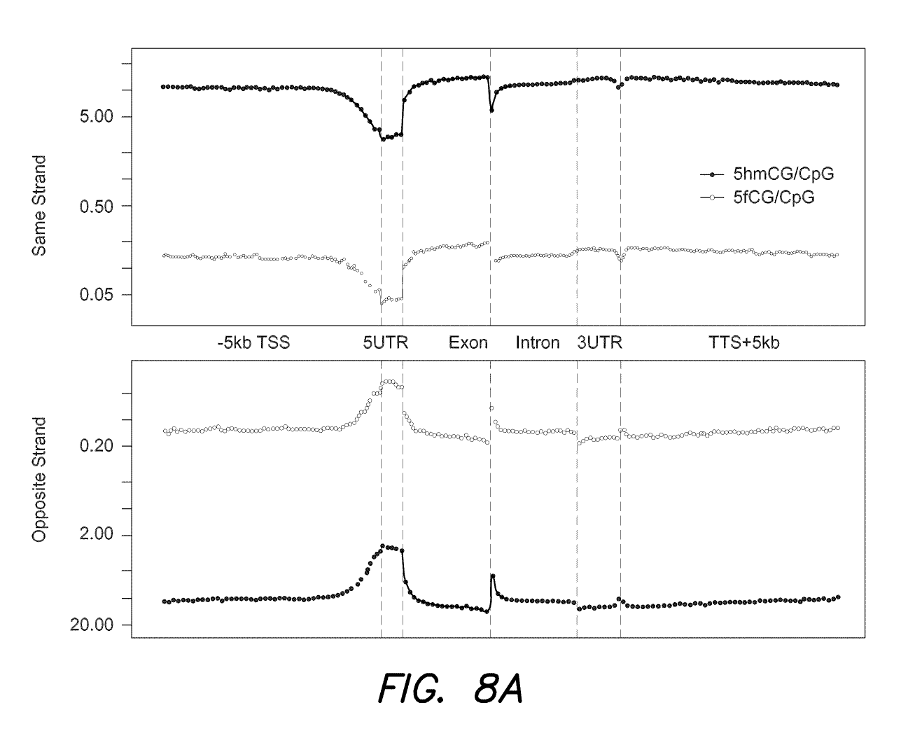

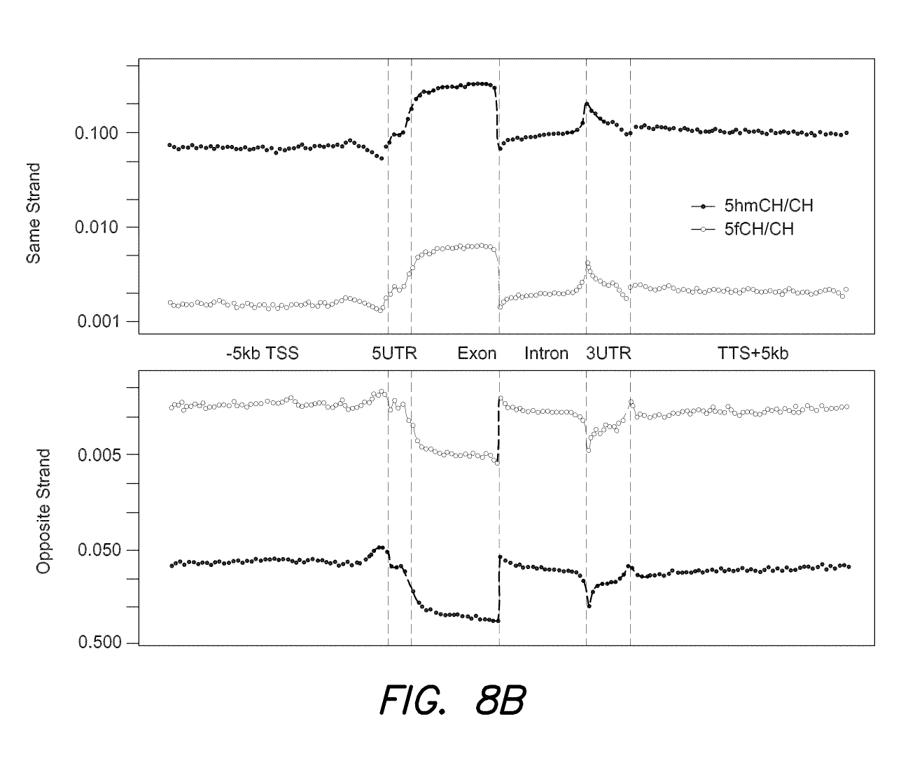

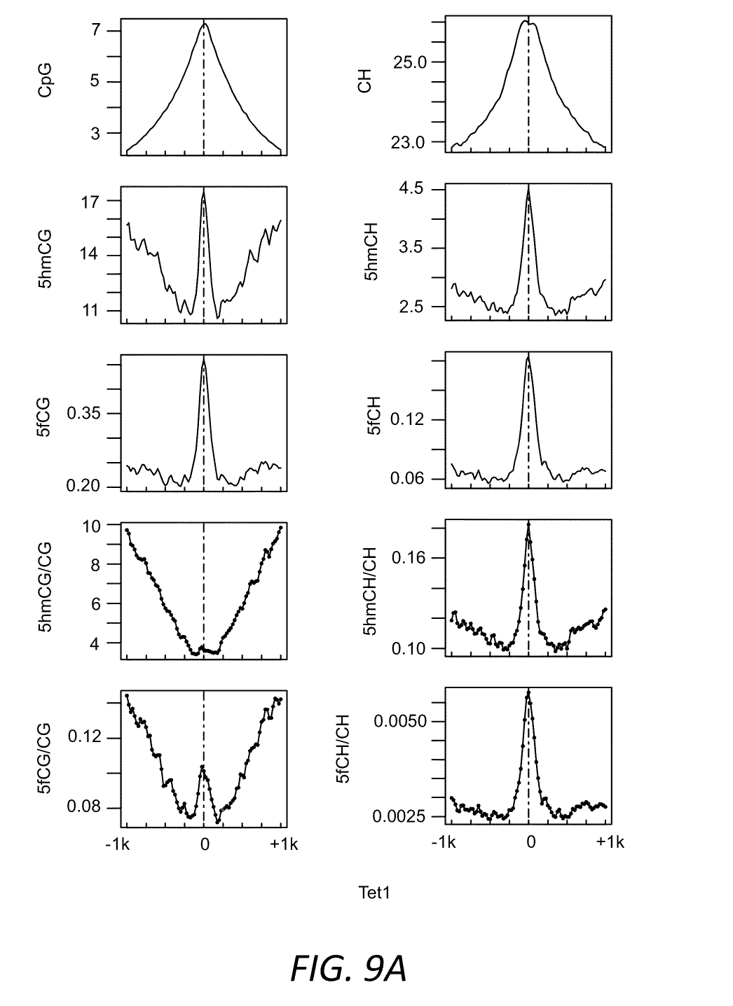

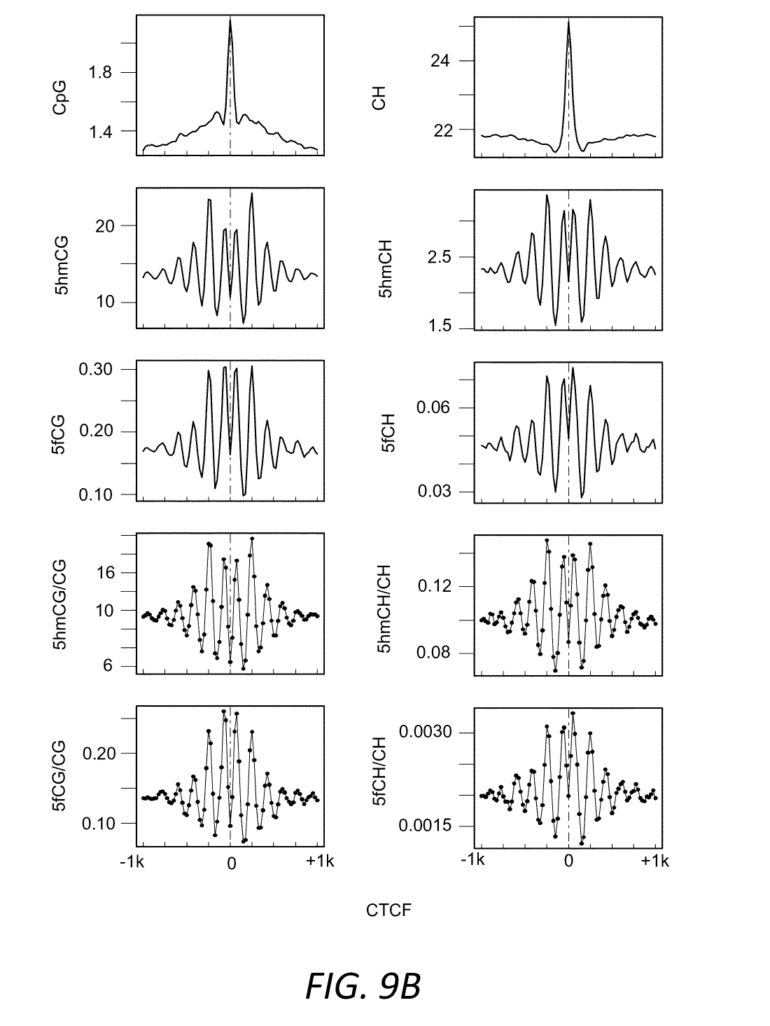

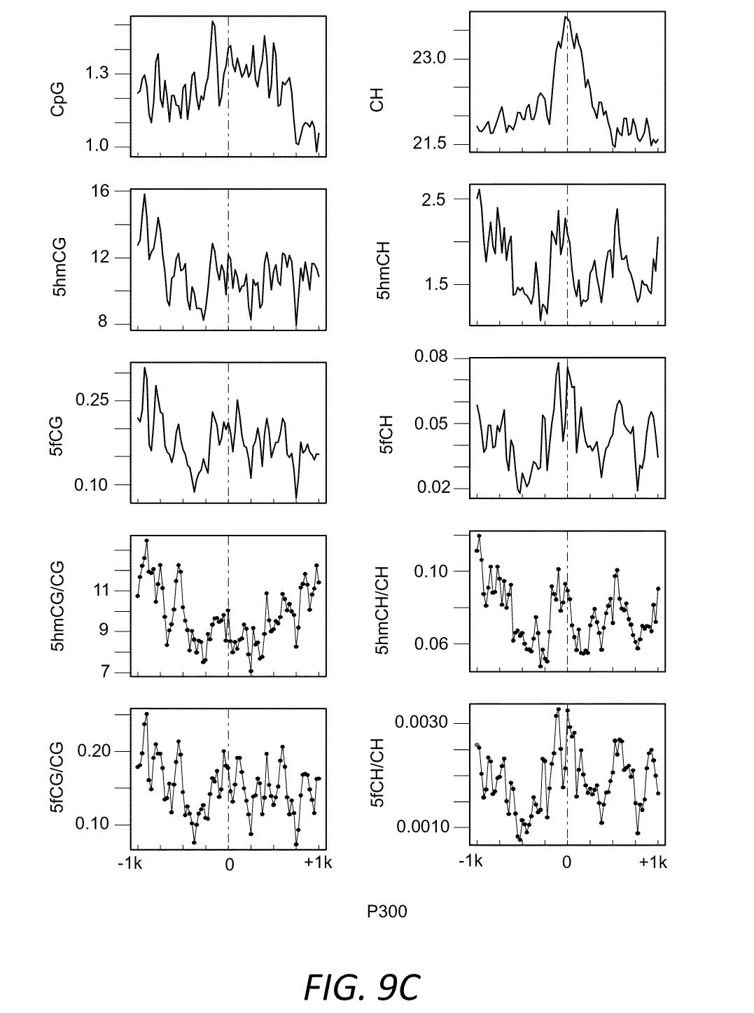

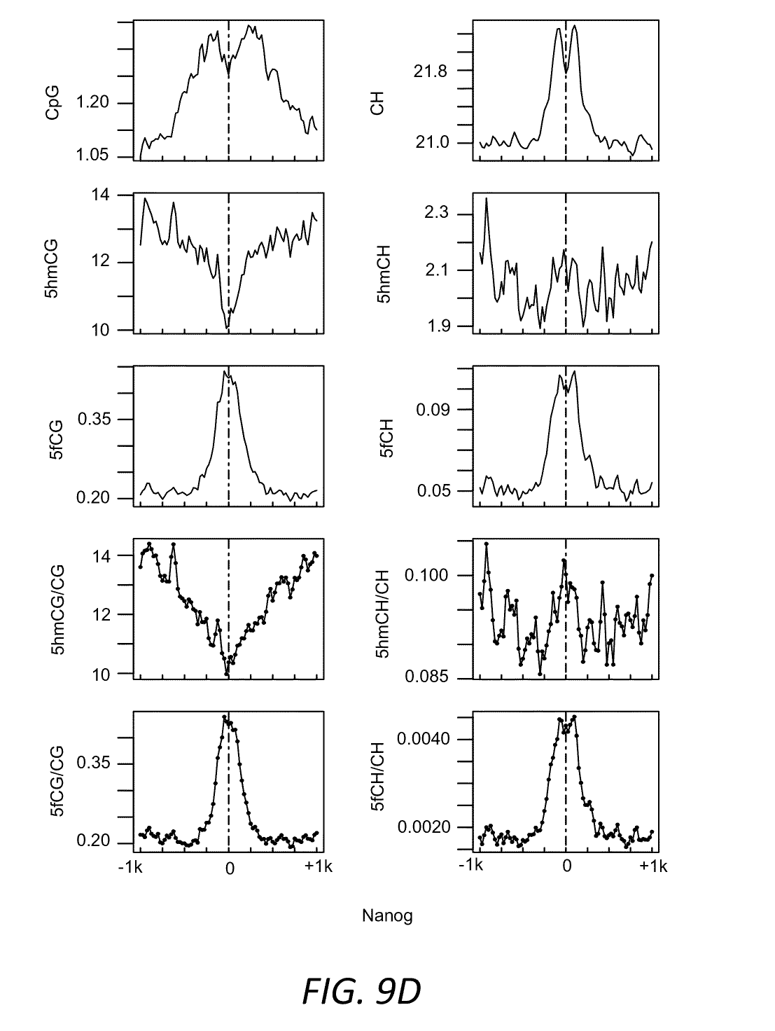

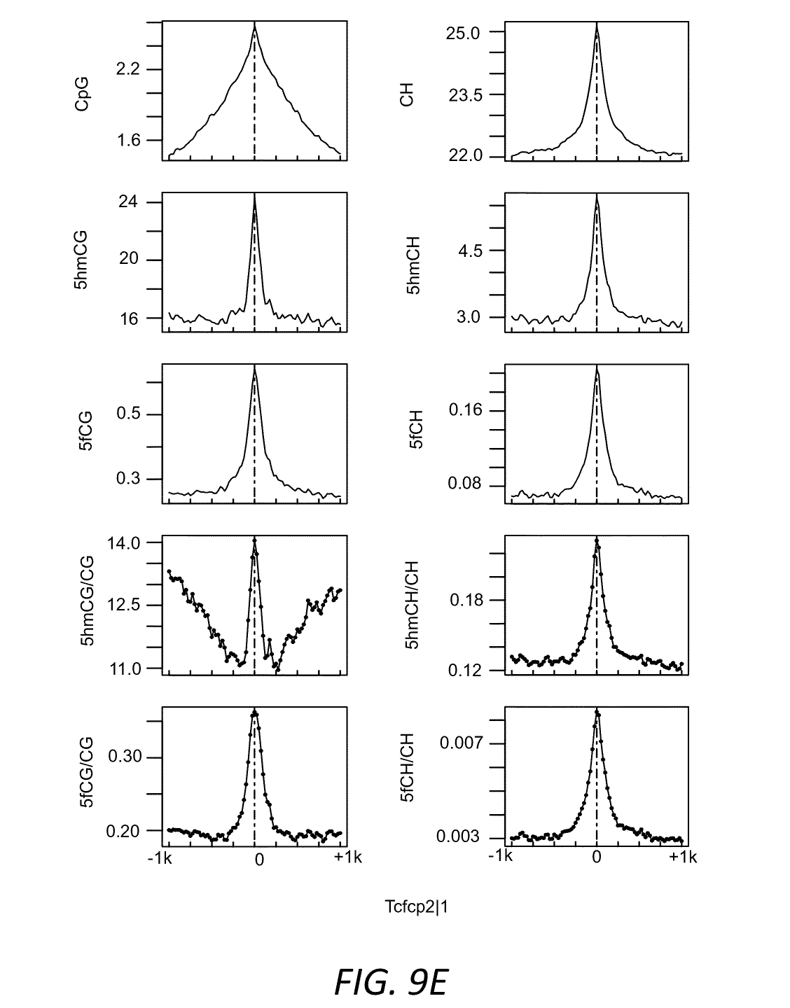

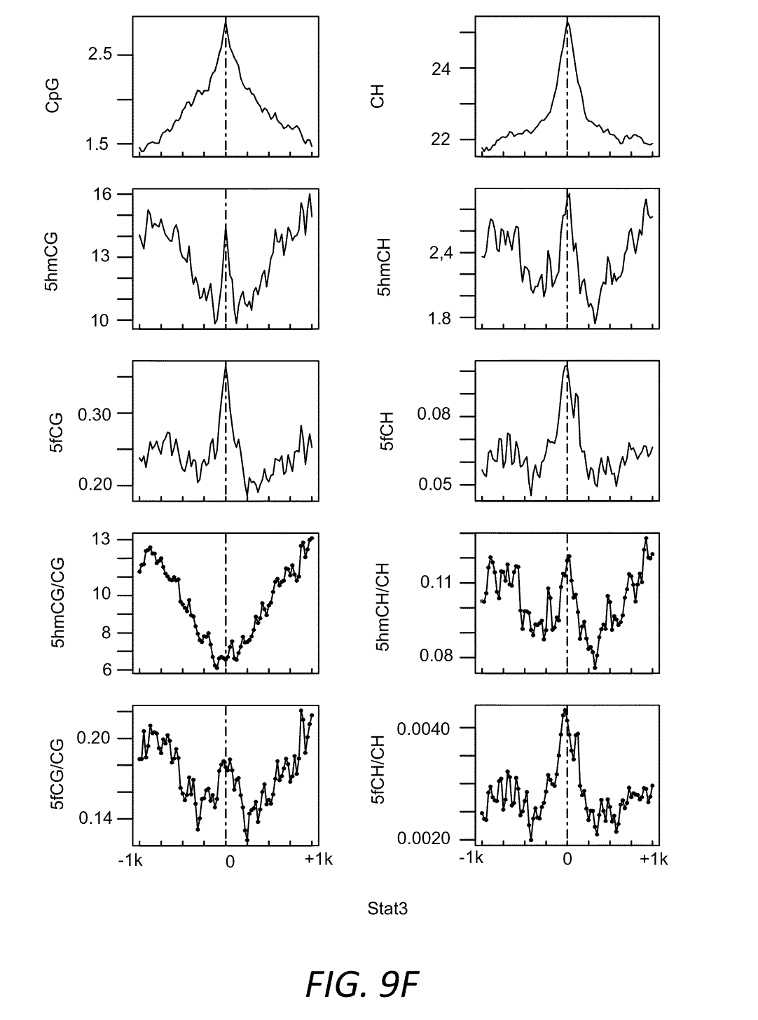

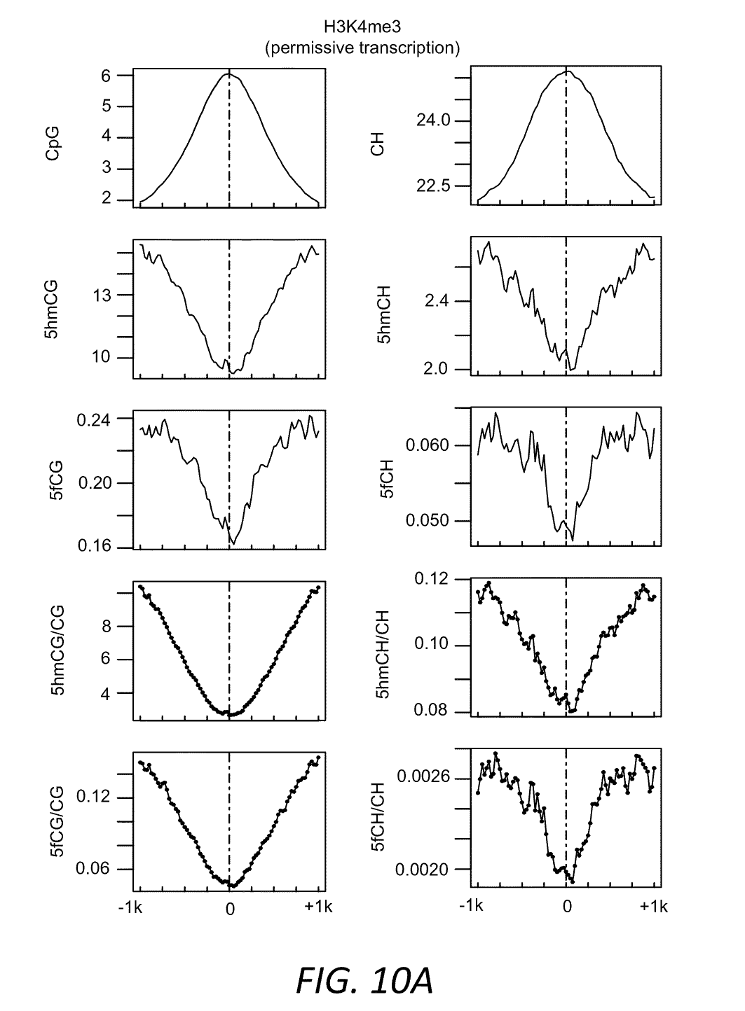

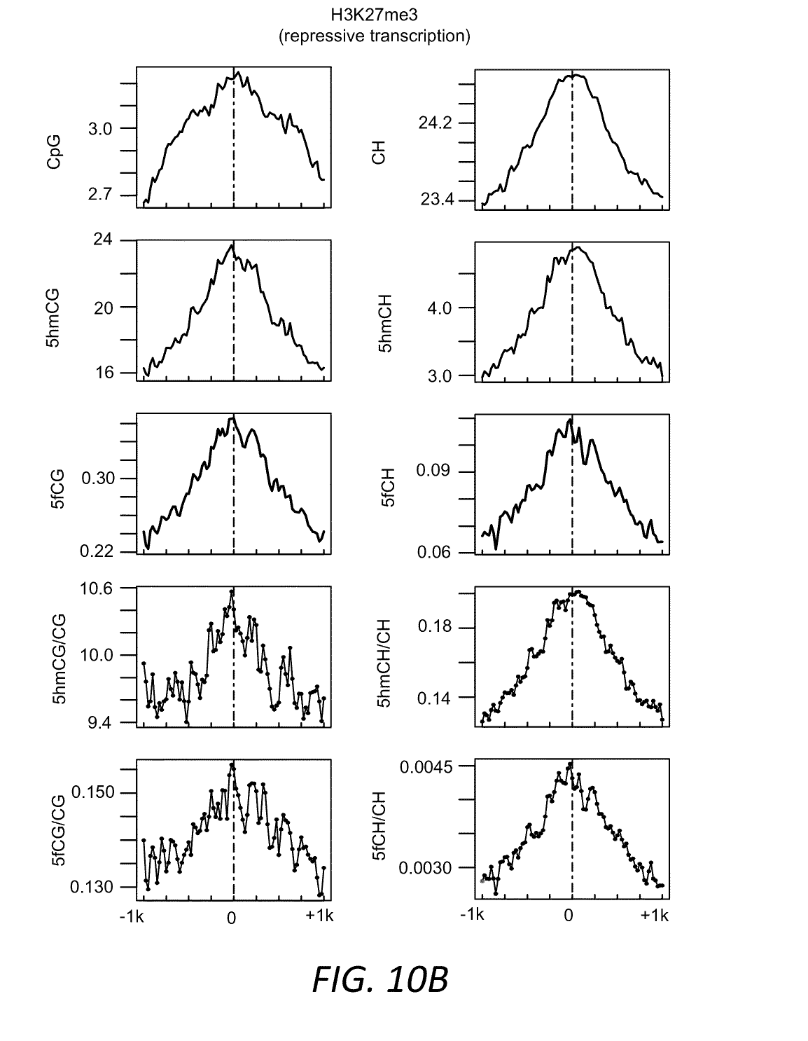

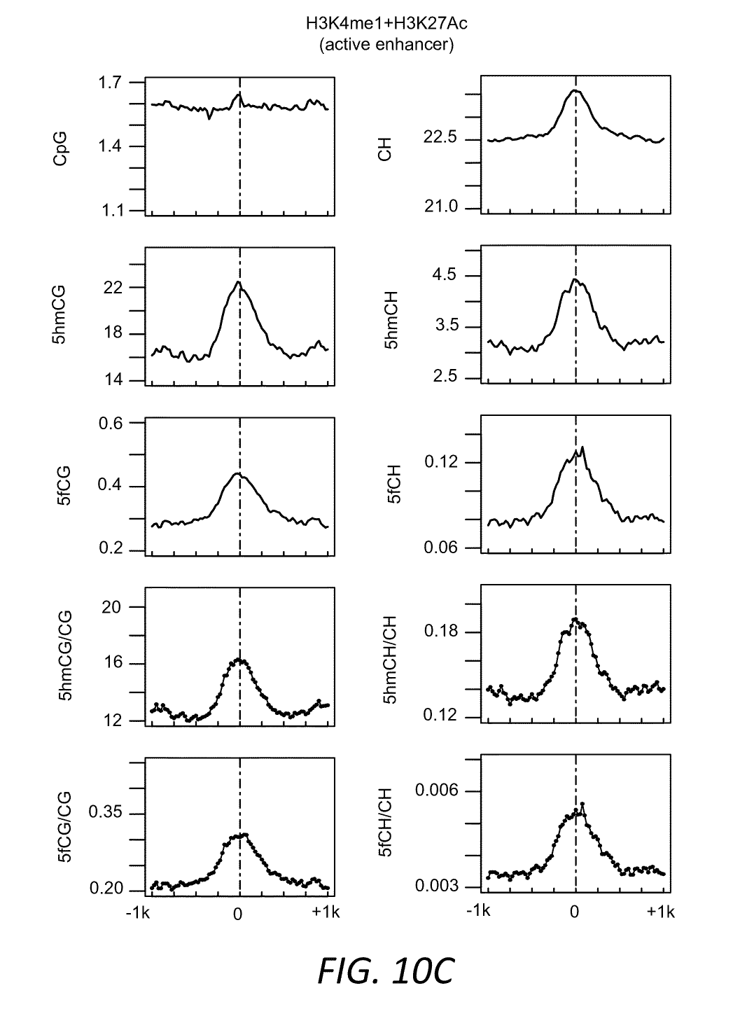

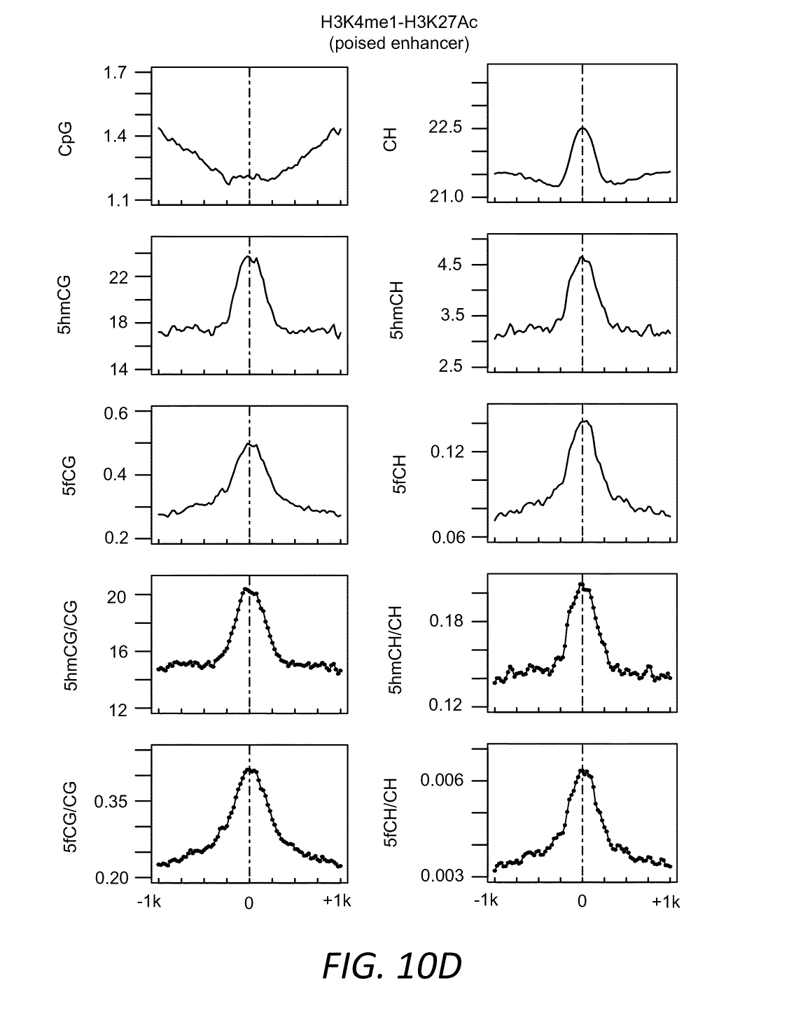

As shown in FIG. 2, genomic DNA can be partially digested with an enzyme that recognizes modified cytosine and cleaves the DNA to generate a single stranded overhang at least at one end and sometimes and both ends of the digested fragment. Those fragments containing 5hmC are a substrate for a glucosyltransferase such as BGT which adds a label permitting the fragments to bind to a solid substrate through a second molecule. In this way only 5hmC containing digested DNA is enriched and methylated cytosine and cytosine containing DNA fragments are removed in the eluent. Adapters can be added to each end of the DNA after restriction endonuclease digestion and before or after subsequent steps leading to enrichment. Adapter ligated DNA can be amplified and subsequently sequenced. In this way, the modified cytosine can be mapped within the fragment based on the knowledge of the cleavage site of the enzyme used for digestion of the genomic DNA. In addition, an average copy number can be obtained from the reads for 5^{hm}CG and/or 5^{hm}CH which reflects the consistency in which the particular modification occurs in the genomic population obtained from a single sample. In addition, multiple libraries each from different samples can be compared for determining biological variability.

Embodiments of the method and compositions utilize one or more enzymes selected from the following: (a) an enzyme that is capable of cleaving DNA containing 5hmC preferably without any further sequence requirements at or downstream of the recognition site such as PvuRts1I or variants thereof; (b) an enzyme that is capable of cleaving DNA containing 5hmC but has limited sequence requirements downstream or upstream of the recognition site such as AbaSI or other members of the XXYZ family or variants thereof (see U.S. Pat. No. 8,969,061); and/or (c) an enzyme that recognizes a specific nucleotide sequence containing 5hmC and cleaves within that sequence, for example MspI or variants thereof.

In these examples, each enzyme cleaves double stranded DNA containing a modified nucleotide to leave a single strand overhang for ligation of an adapter at the cleavage site where the cleavage site is thus differentiated from the second end of the fragment. In the case of PvuRts1I, cleavage results in a two nucleotide 3′ overhang of random sequence. Other enzymes in the PvuRts1I family, e.g., AbaSDFI, produce a two and three nucleotide 3′ overhangs of random sequence.

In addition to being digested with a PvuRts1I, the genomic DNA may be randomly fragmented to provide a population of fragments in which the majority of the fragments that have a PvuRts1I-generated overhang at one end also have a blunt end at the other. In these embodiments, the sample may be fragmented (either before or after digestion by PvuRts1I) to produce fragments of a desired size (e.g., fragments in the range of 100-500 bp) using physical cleavage methods (e.g., sonication, nebulization, or shearing), chemically, or enzymatically (e.g., using a nuclease or transposase). This allows the ends of the fragments to be ligated to different adaptors. In some embodiments, the sample is fragmented after ligation of the adaptor to the PvuRts1I-generated overhang. After fragmentation, the ends can be polished, if necessary, and ligated to the second adaptor using any convenient technique (e.g., by dA-tailing and TA ligation).

The genomic DNA analyzed using the method may be from any source, including, but limited to, a eukaryote, a plant, an animal (e.g., a reptile, mammal, insect, worm, fish, etc.), tissue samples, and cells grown in culture, e.g., stem cells and the like. In particular embodiments, the genomic DNA analyzed using the method may be from a mammalian cell, such as, a human, mouse, rat, or monkey cell.

In addition to one or more cleavage enzymes of the type described in any of (a)-(c) above, a glucosyltransferase can be used to further modify the 5hmC such as but not limited to T4-BGT for reacting a glucose, azido glucose or glucosamine (U.S. Pat. No. 9,200,260) with the 5hmC in DNA to form 5ghmC, 5-azido-ghmC or 6-aminoglucose modified 5hmC (5gnhmC). This provides a chemically reactive site for use in differentiating the 5hmC from other modified nucleotides that do not react with a chemically reactive group. The chemically reactive group may optionally react with a suitable label or affinity tag of the type known in the art to permit enrichment of the modified nucleotide by affinity binding directly or indirectly to a substrate such as a bead, column, multiwall dish, or two dimensional surface that may be suitably coated with an additional molecule for binding the affinity tag. The type of immobilization for enrichment may be selected and/or designed to facilitate subsequent NextGen sequencing.

In one embodiment, where the cleavage enzyme can cleave substantially all of the 5hmC without downstream sequence requirements. it may be used in a molar ratio of cleavage enzyme to 5hmC in eukaryotic DNA of at least 0.25:1, 0.5:1, 0.75:1, 1:1, 5:1, 10:1, 20:1, 30:1, 40:1, 50:1, 60:1, 70:1, 80:1, 90:1, 100:1, 125:1, 150:1, 175:1 or 200:1.

For example, at high concentrations of enzyme, PvuRts1I can recognize a single 5hmC and efficiently cleave at the specified distance DNA downstream of that nucleotide (FIGS. 1A and 1B). In addition to cleavage adjacent to 5hmC, the enzyme also has partial cleavage activity adjacent to 5mC or C. The cleavage products arising from these reactions are washed away as only glucosyltransferase modified 5hmC can be immobilized.

Embodiments of the present methods provide a sensitive, non-biased 5hmC or 5fC single-base-resolution sequencing. The addition of enzymes as described herein permit the localization of individual 5hmC or 5fC and provided genome-wide 5hmC or 5fC mapping at single-base resolution in a method which proved highly specific, sensitive and unbiased.

Ligation of a nucleic acid adapter to one end or to the second end of a cleaved DNA fragment may be performed by standard ligation protocols (New England Biolabs, Inc. 2013-2014 catalog). The nucleic acid adapter may be a double stranded synthetic DNA oligonucleotide with a single strand overhang of 2 or more NN for hybridizing to the 3′ overhang at the end of the DNA strand containing 5hmC. The non-hybridizing end of the adaptor may lack a phosphate group to prevent self-ligation.

Where 5hmC is present on one strand of a duplex DNA, the cleavage fragment will have a single strand overhang at the 3′ end only. The 5′ end of the same strand will have a blunt end with the second strand of the duplex to which a second synthetic oligonucleotide adapter may be ligated. If 5hmC occurs at a position adjacent to a G sequence and is found on opposing strands of the genomic fragment then single strand overhangs will occur at both ends of the cleaved genomic fragment. Under these circumstances, the genomic DNA fragment having adapters with single strand overhands at both ends can be repaired to form a continuous DNA molecule using for example, Taq ligase and optionally a flap endonuclease prior to amplification (see for example, U.S. Pat. No. 7,700,283 and U.S. Pat. No. 8,158,388).

Alternatively, the eukaryotic genome may be randomly fragmented, amplified through 1-2 rounds of amplification to replace duplexes with 5hmC on both strands at a CpG with duplexes with a 5hmC on one strand only, cleaved with a PvuRts1I family enzyme and then reacted in the methods described herein namely, glucosylated, first adaptor ligated, enriched for 5hmC containing duplexes, second adaptor ligated and amplified for sequencing. Alternatively phiX whole genome amplification may be utilized prior to cleavage with a PvuRts1I family of enzymes.

Examples of alternative enrichment protocols that may be used in addition or instead of substrates for a glucosyltransferase include: treatment of 5hmC with a 5hmC antibody or sodium bisulphate to form cytosine 5-methylenesulphonate (CMS) where immobilized anti-CMS binds to CMS for enrichment of 5hmC containing molecules; or using a glucosyltransferase, with glucosamine for reaction with 5hmC, followed by linkage of an NHS-biotin group to glucosamine to form biotin-glucosamine-hmC for enrichment of 5hmC; or use of a glucosyltransferase, and sodium periodate for cleavage of the vicinal hydroxyl group on 5ghmC or 5gnhmC forming an aldehyde groups and hydroxylamine-biotin group can be used to react with aldehyde group to enrich 5hmC; or a J-binding proteinl (JBP-1) can specifically bind to 5gmC, so SNAP-JBP1 can be produced to enrich 5hmC.

The affinity matrix may be a bead such as a magnetic bead, column, paper, coated plastic or other solid surface suitable for immobilizing an affinity molecule bound to a nucleic acid of interest. The matrix may comprise streptavidin, chitin, amylose, protein A, a modified benzyl guanine, receptor agonist or antagonist or other suitable matrices for binding the affinity label such as biotin, chitin binding domain, maltose binding domain or mutants thereof, antibodies or portions thereof, SNAP-Tag® (New England Biolabs, Ipswich, Mass.) or receptor agonist or antagonist.

A distributed alignment tool that combines BWA was described by Li and Durbin, 2009, Bioinformatics 2009; 25:1754-1760 that utilizes duplicate read detection and removal and harnesses the Hadoop MapReduce framework to efficiently distribute I/O and computation across cluster nodes and to guarantee reliability by resisting node failures and transient events such as peaks in cluster load. This method was used here to achieve pair-end alignment of sequences read by Illumina sequencing machines using a version of the original BWA code base (version 0.5.8c) that has been refactored to be modular and extended to use shared memory to significantly improve performance on multicore systems.

Uses of embodiments of the methods described herein include genome-wide 5hmC mapping in cancer cells. Loss of 5hmC has been considered as a signature for various cancer cells, including lung, brain, breast, melanoma (Lian, et al., (2012); Kudo, et al., (2012); Jin, et al., (2011)).

However, the actual function of 5hmC during tumor progression is still unknown. To understand the function of 5hmC in cancer, it is important to pinpoint their location in the genome at single-base resolution, which is challenging due to the low abundance of 5hmC. The methods described herein and exemplified in

FIG. 2 can be used to identify the 5hmC distribution between cancer cells and their untransformed non-tumorigenic cells more accurately than previously was possible.

Another use of embodiments includes genome wide mapping of 5fC at single-base resolution. Accurate 5fC single-base sequencing methods can significantly enhance an understanding of the biological function of demethylation intermediates. 5fC can be efficiently removed by TDG in vivo, the distribution of 5fC can unveil the “hot spot” of active demethylation. 5fC may function as a gene regulator just as 5hmC does.

All references cited herein including U.S. provisional Ser. No. 61/864,299 are incorporated by reference.

### EXAMPLES

Aspects of the present teachings can be further understood in light of the following examples, which should not be construed as limiting the scope of the present teachings in any way.

### Example 1

### Characterization of 5hmC-dependent PvuRts1I

TABLE 1
Synthetic oligonucleotides containing 5hmC used to characterize
PvuRts1I

| Name | Sequence |
|---|---|
| 5hmC_21C_top | GGTTGGACTCAAGACGATA(5hmC)TTACCGGATAAG |
| | GCGCAATTAGATTACTTAACCT (SEQ ID NO: 1) |
| 5mC_21C_top | GGTTGGACTCAAGACGATA(5mC)TTACCGGATAAGG |
| | CGCAATTAGATTACTTAACCT (SEQ ID NO: 2) |
| 5hmC_21_5hmC_bottom | AGGTTAAGTAAT(5hmC)TAATTGCGCCTTATCCGGTA |
| | AGTATCGTCTTGAGTCCAACC (SEQ ID NO: 3) |
| 5hmC_21C_mC_bottom | AGGTTAAGTAAT(5mC)TAATTGCGCCTTATCCGGTAAGTATCG |
| | TCTTGAGTCCAACC (SEQ ID NO: 4) |
| 5hmC_21C_bottom | AGGTTAAGTAATCTAATTGCGCCTTATCCGGTAAGTATCGTCTT |
| | G AGTCCAACC (SEQ ID NO: 5) |
| 5hmC_nonC_top | GGTTGGACTCAAGACGATA(5hmC)TTACCGGATAAGGCGCAATTA |
| | TATTACTTAACCT (SEQ ID NO: 6) |
| 5hmC_nonC_bottom | AGGTTAAGTAATATAATTGCGCCTTATCCGGTAAGTATCGTCTTG |
| | AGTCCAACC (SEQ ID NO: 7) |
| C_21C_top | GGTTGGACTCAAGACGATACTTACCGGATAAGGCGCAATTAGATT |
| | ACTTAACCT (SEQ ID NO: 8) |

Equal volumes of top strand and bottom strand were mixed to provide a final concentration of double-stranded substrate is at 5 μM. In Table 1, 5hmC_21C_top pairs with 5hmC_21_5 hmC_bottom as substrate hmC/hmC. Similarly, 5hmC_21C_top pairs with 5hmC_21_mC_bottom as substrate hmC/mC; 5hmC_21C_top pairs with 5hmC_21C_bottom as substrate hmC/C; 5hmC_nonC-top pairs with 5hmC_nonC_bottom as substrate hmC/nonC; 5mC_21C_top pairs with 5hmC_21_mC_bottom as substrate mC/mC; 5mC_21C_top pairs with 5hmC_21_C_bottom as substrate mC/C; 5hmC_nonC_top pairs with 5hmC_nonC_bottom as substrate C/C. To characterize the property of PvuRts1I, 0.1 μl of each substrate was incubated with 2 μl of serial dilution of PvuRts1I (the highest concentration is 110 ng/μl) at room RT for 2 hours. Then the reaction mix was resolved in 10% TBE gel, as shown in FIG. 1B. At the highest concentration shown in lane 1 for each sample, PvuRts1I exhibits similar activity on substrates hmC/hmC, hmC/mC, hmC/C and hmC/nonC. Although PvuRts1I can still partially digest 5mC-5mC, 5mC-C or even C-C, these non-specific digestions will not affect the ultimate 5hmC library after enrichment by Seal. The results are shown in FIG. 1A-1B.

### Example 2

### Library Construction for Sequencing and Mapping

TABLE 2
Oligonucleotides used in 5hmC mapping

| Name | sequences |
|---|---|
| Adapter | ACA CTC TTT CCCTAC ACG ACG CTC TTC |
| P1 top | CGATCT NN (SEQ ID NO: 9) |
| Adapter | AGATCG GAA GAG CGT CGT GTA GGG AAA |
| P1 bottom | GAGTGT (SEQ ID NO: 10) |
| Adapter | /5Phos/GAT CGG AAG AGC ACA CGT CTG |
| P2 top | AACTCC AGT C (SEQ ID NO: 11) |
| Adapter | GACTGG AGT TCA GAC GTGTGC TCT TCC |
| P2 bottom | GAT CT (SEQ ID NO: 12) |

(a) 5hmC Library Construction:

The E14 cells were cultured as previously described (Sun, et al., Cell Rep, 3:567-576 (2013)). E14 genomic DNA was extracted with a Qiagen DNeasy Blood and Tissue Kit (Qiagen, Valencia, Calif.). To generate the 5hmC library, 2 μg of genomic DNA was digested with 0.7 μg of PvuRts1I at RT for 2 hours. Next, 30 units of T4-BGT (New England Biolabs, Ipswich, Mass.) and 75 μM UDP-6N3-Glc were added to the reaction and incubated at 37° C. for 2 hours. DNA ends digested by PvuRts1I were ligated with 7 μM Adapter P1 (top: ACACTCTTTCCCTACACGACGCTCT TCCGATCTNN (SEQ ID NO:9) and bottom: AGATCGGAAGAG CGTCGTGTAGGGAAAGAGTGT (SEQ ID NO:10)) with T4 DNA ligase at 16° C. overnight. The next day, genomic DNA was sheared to around 200 bp by the Covaris s-series sonicator (Covaris, Woburn Mass.) according to the suggested settings. The sheared genomic DNA was then purified with DNA Clean and Concentrator kit (Zymo, Irvine, Calif.). The purified DNA was reacted with 1 mM dibenzocyclooctyne-S-S-PEG3-biotin conjugate (Click Chemistry Tools, Scottsdale, Ariz.) at 37° C. for 2 hours. The DNA was then purified again with DNA Clean and Concentrator kit. Subsequently, streptavidin beads (New England Biolabs, Ipswich, Mass.) were added to the DNA and rotated at RT for 2 hours. The DNA captured on the beads was washed twice with 5 mM Tris pH=8.0 and 1M NaCl. After that, the DNA was end repaired and dA-tailed with NEBNext® End Repair and NEBNext® dA-Tailing Module (New England Biolabs, Ipswich, Mass.), respectively. Subsequently, 1 μM adapter P2 (top: /SPhos/GATCGGAAG AGCACACGTCTGAACTCCAGTC (SEQ ID NO:11) and bottom: GACTGGAGTTCAGACGTGTGCTCTTCC GATCT (SEQ ID NO: 12)) was added to perform ligation with T4 DNA ligase at RT for overnight. Lastly, 100 mM DTT was added to the reaction to cleave the disulfide bond in order to release the 5hmC library from the biotin-streptavidin beads. The released DNA was purified via Ampure® Beads (Beckman Coulter, Indianapolis, Ind.) with the ratio 1:1 to remove unligated adapter P2. The 5hmC library was then amplified with NEB universal primer and NEB indexl primer (New England Biolabs, Ipswich, Mass.) and subject to Next Generation sequencing pipeline. Illumina HiSeq sequencing was performed in Hudson Alpha Institute for Biotechnology. The results are shown in FIG. 3A-3D.

(b) 5fC Library Construction

To ensure the complete conversion of 5hmC to 5gmC, 12 μg of E14 genomic DNA were first incubated with 180 units of T4-BGT and 450 μM UDP-Glc at 37° C. for 3 hours. Then additional 180 units of T4-BGT and 450 μM UDP-Glc were added to the reaction at 37° C. for overnight. After that, fresh additional 180 units of T4-BGT and 450 μM UDP-Glc were added to the reaction at 37° C. for 3 hours. The genomic DNA was purified via phenol/chloroform. After the conversion, 100 mM of NaBH4 was added to the DNA and incubated for 2 hours at RT. Genomic DNA was then precipitated by ethanol and ready for library construction. The results are shown in FIG. 7A-7C.

(c) Analysis of Sequence Data

Sequencing of 5hmC and 5fC library was performed on the Illumina HiSeq platform with single-end 50 bp reads. Briefly, all the raw reads are mapped to the reference genome using the Bowtie aligner (Langmead, et al., Genome Biol, 10(3):R25 (2009)) with parameters (-n 1-I 25--best--strata-m 1), which allows up to 1 mismatch within the first 25 high quality bases and only keeps uniquely mapped reads. The positions where sequencing reads align to the reference genome indicate the enzyme cleavage sites. 5hmC or 5fC sites were expected to be located on the opposite strand 11 to 12 nucleotides downstream of the cleavage sites. For identified 5hmC and 5fC sites, both genomic coordinates and sequence context were recorded. The copy number of a 5hmC or 5fC site is defined as the total number of reads from a particular site and used as an indicator of 5hmC level (see for example, FIG. 3D).

(d) Quantification of 5hmC and 5fC Level by Sequencing Copy Number and LC-MS/MS Measurement

The copy number of individual sites from Pvu-Seal-seq is an indicator of relative 5hmC or 5fC level. In order to compare modification levels between different libraries, the sequencing copy numbers were normalized by both the library size (i.e., total number of 5hmC or 5fC reads) and the global 5hmC or 5fC level measured by LC-MS/MS. The normalization factor F=(total number of 5hmC or 5fC reads)/((LC-MS/MS global 5hmC or 5fC measurement)×(1.0E+8)). The normalized copy number=original copy number/F, and this value was used to compare modification levels between different libraries and between different modification types (i.e., 5hmC vs. 5fC).

(e) Genomic Profiling of 5hmC and 5fC

The genomic distributions of 5hmC and 5fC in gene related regions were investigated by metagene plots (Sun, et al., (2013)). RefSeq gene annotations of mm9 genome were downloaded from UCSC Genome Browser (Fujita, et al., 2011) in February 2012. The 2 kb upstream region of TSS, 5′ UTR, exon, intron, 3′UTR, and 2 kb region downstream of each non-redundant RefSeq gene were divided into equal-sized bins respectively. The background CpG sites, 5hmC and 5fC sites were mapped to each bin of individual regions using the BEDTOOL (Quinlan and Hall, Bioinformatics, 26:841-842 (2010)). Bin-level 5hmC and 5fC levels were calculated as the sum of normalized sequencing copies of all the sites within that bin, and each was also corrected by background CpG density. The means of all the non-redundant RefSeq genes were calculated and were used for the metagene plots. The results are shown in FIG. 8A-8B.

(f) Correlating 5hmC and 5fC to ChIP-Seq Data Sets

The TET1 ChIP-seq data set was downloaded from the GEO database (GSE24843). The peaks of TET1 binding sites were called using the MACS program with the following criteria: peak p value <10-8, fold enrichment over IgG >10.

ChIP-seq data sets of 13 TFs (Nanog, Oct4, STAT3, Smad1, Sox2, Zfx, c-Myc, n-Myc, Klf4, Esrrb, Tcfcp2I1, E2f1, and CTCF) and two transcription regulators (p300 and Suz12) were downloaded from the GEO database (GSE11431) (Chen, et al., Cell, 133:1106-1117 (2008)). The genomic coordinates of the original data sets are based on the mm8 reference genome and so were remapped to the mm9 reference using the LiftOver tool.

ChIP-seq data sets of histone modification marks H3K4me3 and H3K27me3 were downloaded from NCBI GEO database (GSE12241) (Mikkelsen, et al., Nature, 448:553-560 (2007)). ChIP-seq data sets of two enhancer histone mark H3K27ac and H3K4me1 were downloaded from NCBI GEO database (GSE24165) (Creyghton, et al., Proc Natl Acad Sci USA, 107:21931-21936 (2010)). Sequencing reads (mm8) to mm9 were remapped via the LiftOver tool and then used the MACS program (peak p value <10-5, fold enrichment over control H3 >10) to identify genomic intervals enriched with a specific chromatin mark.

−1 kbp to +1 kbp regions of the identified peak summits or binding centers were extracted and binned into 50 bp sliding windows at 25 bp steps. 5hmC, 5fC and CpG sites were mapped to individual bins. The total number of normalized 5hmC or 5fC reads were normalized to the bin lengths as well as to the background CG or CH densities. The means of all the peaks were used to generate the trend plots. The results are shown in FIGS. 9A-9F and 10A-10D.

### Example 3

### Sequencing Results from the 5hmC Library from Mouse ESC E14 Genomic DNA

The raw reads were mapped to the mouse reference genome (UCSC mm9) using the Bowtie aligner (Langmead, et al., (2009)) with parameters (-v 2--best--strata-m 1), which allows up to 2 mismatches within the 50 bases and only keeps uniquely mapped reads. The positions where sequencing reads align to the reference genome indicate the enzyme cleavage sites. 5hmC sites are expected to be located on the opposite strand 11 to 12 (or 11-13) nucleotides downstream of the cleavage sites. For identified 5hmC sites, both genomic coordinates and sequence context are recorded. The copy number of a 5hmC site is defined as the total number of reads from a particular site and used as an indicator of 5hmC level.

From one lane of HiSeq, we obtained 166.9 million reads, of which 130.3 (78%) million reads were unambiguously mapped to the reference genome. The weblogo showed that substantially all of the reads contains C at 12/13 position, which confirms PvuRts1I cutting (FIG. 3A). Within mapped reads, 122.6 million of 5hmC containing reads (94%) were called, corresponding to 24.9 million unique 5hmC sites.

In contrast, TAB-seq only detected 2 million unique 5hmC sites with a sequencing depth of 17.6× per cytosine, which requires approximately more than 5 lanes of Illumina HiSeq. In the mouse ESC genome, nearly 25% of methylation was in a non-CG context (mCH, H=A,C or T) (Lister, et al., Nature, 462(7271):315-22 (2009)). ^{m}CH has also been found in mouse brain genome and accumulates in neurons during fetal to young adult development, which suggests an important role of mCH during brain development (Xie, et al., Cell, 148(4):816-31 (2012); Lister, et al., Science, 341(6146) (2013); Kinney, et al., J Biol Chem, 286(28):24685-93 (2011)).

The existence of 5hmCH has been reported in mouse ECS cells (Ficz, et al., (2011)). However, TAB-seq detected only 1.3% of 5^{hm}CH sites (FIG. 4A). In Pvu-Seal-seq data, we detected 33.8% of 5^{hm}C exists in non-CG context. We determined that the 5hmCG sites have higher average copy numbers (mean=6) than the 5^{hm}CH sites (mean=2.7).

To confirm that PvuRts1I did not require the second C for efficient cutting, the reads were divided into two groups: 1) C-C, which contains two Cs flanking the cutting site; 2) C-nonC, which contains no Cs at position 9-11 downstream of the cutting site. The C-C and C-nonC make up for 76% and 24% of the total reads, which is similar to TAB-seq data with 74.5% of C-C and 25.5% of C-nonC (FIG. 4B). These data showed that PvuRts1I recognized every single 5hmC and did not require the second C for efficient cutting.

TABLE 3
Overlapping ratio of 5^{hm}CG, 5^{hm}CH, 5^{f}CpG and 5^{f}CH sites with different
copy numbers. (Copy number 6: gives 97% = reproducible results)

| 5^{hm}CG | | | | 5^{hm}CH | | | |
|---|---|---|---|---|---|---|---|
| Copy | non- | | overlapping | Copy | non- | | overlapping |
| number>= | overlap | overlap | ratio | number>= | overlap | overlap | ratio |
| 2 | 1763334 | 13421959 | 88.4% | 2 | 2816852 | 3307872 | 54.0% |
| 3 | 928402 | 11319926 | 92.4% | 3 | 1035960 | 2302636 | 69.0% |
| 4 | 497802 | 9576657 | 95.1% | 4 | 403313 | 1648194 | 80.3% |
| 5 | 272355 | 8136264 | 96.8% | 5 | 171213 | 1207003 | 87.6% |
| 6 | 151659 | 6943085 | 97.9% | 6 | 78673 | 899254 | 92.0% |
| 5^{f}CG | | | | 5^{f}CH | | | |
| Copy | non- | | overlapping | Copy | non- | | overlapping |
| number>= | overlap | overlap | ratio | number>= | overlap | overlap | ratio |
| 2 | 4211632 | 4124985 | 49.5% | 2 | 6002375 | 1322846 | 18.1% |
| 3 | 3474169 | 3666383 | 51.3% | 3 | 4737146 | 1142859 | 19.4% |
| 4 | 2863694 | 3259188 | 53.2% | 4 | 3732107 | 985722 | 20.9% |
| 5 | 2342025 | 2889261 | 55.2% | 5 | 2897531 | 846141 | 22.6% |
| 6 | 1897441 | 2552144 | 57.4% | 6 | 2209501 | 722474 | 24.6% |
| 7 | 1523933 | 2250662 | 59.6% | 7 | 1654063 | 614928 | 27.1% |
| 8 | 1217024 | 1983008 | 62.0% | 8 | 1222617 | 522058 | 29.9% |
| 9 | 970337 | 1747940 | 64.3% | 9 | 896197 | 443540 | 33.1% |
| 10 | 773160 | 1542889 | 66.6% | 10 | 654777 | 377092 | 36.5% |

TABLE 4
Quantification of 5^{m}C/C, 5^{hm}C/C and 5^{f}C/C ratio in the genomic
DNA of E14 by LC-MS/MS. The amount of C was set to 106, and the
amount of 5mC, 5hmC and 5fC were calculated in E14 genomic DNA.
The experimental row is determined experimentally and reference
row is from the data reported before (Ito, et al., 2011). The
results confirm the rarity of occurence of 5hmC and 5fC.

| | | 5^{m}C | 5^{hm}C | 5^{f}C |
|---|---|---|---|---|
| E14 DNA | C | (5^{m}C/C) | (5^{hm}C/C) | (5^{f}C/C) |
| Exp. | 10⁶ | 33600 | 1490 | 20.2 |
| | | (3.36%) | (0.149%) | (0.00202%) |
| Ref. | 10⁶ | 29600 | 1120 | 17.9 |
| | | (2.96%) | (0.112%) | (0.00179%) |

Table 4: Quantification of 5mC/C, 5^{hm}C/C and 5^{f}C IC ratio in the genomic DNA of E14 by LC-MS/MS. The amount of C was set to 106, and the amount of 5mC, 5hmC and 5fC were calculated in E14 genomic DNA. The experimental row is determined experimentally and reference row is from the data reported before (Ito, et al., 2011). The results confirm the rarity of occurrence of 5hmC and 5fC.

### Example 4

### Genome Mapping of 5hmC in E14 Genomic DNA

The overall strategy is illustrated in FIG. 5. First, the genomic DNA is treated with T4-BGT and UDP-Glc to convert all 5^{hm}Cs to 5^{gm}C (100%). Then, NaBH₄ is used to reduce the 5fC to 5hmC. After these treatments, the genome only contains 5hmC, 5gmC, 5mC and C; therefore we can apply Pvu-Seal-seq to pinpoint 5hmC (converted from 5fC) at single-base resolution.

### Example 5

### Reduction of 5fC to 5hmC by NaBH₄

To investigate the condition of 5fC reduced to 5hmC by NaBH₄, a 1.6 kb PCR products was generated with all Cs replaced by 5fC. Different concentrations of NaBH₄ were incubated with this substrate at RT for 1 hour. The product was broken down into nucleosides (DNA degradase provided by Zymo (Irvine, Calif.)) and was subjected to LC/MS analyses. As shown in FIG. 6, 100 mM NaBH₄ can convert 5fC to 5hmC with an efficiency close to 100%. Using this methodology, it the dynamics of 5fC during E14 cell differentiation can be studied.

### Example 6

### Determining the Presence and Fate of 5fC in Stem Cells

Upon the withdrawal of LIF from E14 stem cell cultures, E14 cells are differentiated to embryoid bodies, during which 5hmC levels first increase then slowly decrease, whereas 5mC levels increase gradually over time (Kinney, et al., (2011)). The dynamics of 5fC appearance and disappearance during this process is indicative of hotspots of demethylation which reveal the relationship between demethylation, transcription and differentiation. Genome-wide 5fC sequencing can be performed to sequence 5fC at single-base resolution at different time points of E14 differentiation.

### DISCUSSION OF THE FIGURES

The resolution of embodiments of the methods described herein are significantly improved over methods in the prior art. Sequencing data shows that 94% of the detected 5hmC containing fragments contained the expected cytosine. 76% of the 5hmC sites were in CpG context and the remaining 24% of 5hmC sites were in CH context (see FIGS. 3A-3D).

Each library was sequenced on Illumina HiSeq platform (one lane) and produced 263 million (13.2 Gbp) and 266 million (13.3 Gbp) raw reads respectively. 74% of the reads from each replicate could be uniquely mapped to the mouse reference genome (mm9). Among all the uniquely mapped reads, 94% contained the expected cytosine (11 or 12 nt away from the cutting site, FIG. 3A), resulting in 32.1 and 33.1 million predicted 5hmC sites from the two replicates respectively. Between the two replicates, 65% of the 5hmC sites (20.8 million) were overlapping. Among the overlapping 5hmC sites, 76% of the 5hmC sites were in CpG context and the remaining 24% of 5hmC sites were in CH context (17% in CHH and 7% in CHG, where H=A, C or T) (FIG. 3B). The overlapping ratio of 5hmCG sites (82%) was much higher than that of the 5hmCH sites (38%) (FIG. 3C). The copy numbers (defined by sequencing read counts) of the 5hmC sites, which could be used as an indicator of relative 5hmC level, were highly correlated between the replicates (Pearson's correlation=0.99; Spearman's rank correlation=0.71), indicating high reproducibility of the method. On average the overlapping 5hmC sites had significantly higher copy number (6.3) than the non-overlapping sites (1.7) (Student's T test, P-value <1.0E-6) (FIG. 3D). Also, the average copy number of 5hmCpG sites is significantly higher than that of the 5hmCH sites for both overlapping sites and non-overlapping sites (FIG. 3D).

In one example, two 5fC libraries from the same batch of E14 genomic DNA were used for 5hmC library constructions and detected 19.4 and 16.5 million unique 5fC sites, respectively (FIG. 7A-7C). Among these, 6.2 million (38%) sites were overlapping. Among the overlapping 5fC sites, 75% of them were in CpG context and 25% of them were in CH context (17% is in CHH and 8% is in CHG) (FIG. 7A). Similar to 5hmC, overlapping 5fC sites had significantly higher average copy number (8.6) than non-overlapping sites (4.7) (Student's T test, P-value <1.0E-6). The 5fCpG sites had significantly higher average copy number (7.1) than the 5fCH sites (4.8), and the overlapping ratio of 5fC in CpG context (55%) was significantly higher than that in CH context (20%) (FIG. 7B). However, upon investigating the 5fC regional density in 100 bp sliding windows across the entire genome, there was a meaningful correlation between the two libraries, especially at regions with high 5fC level (Pearson correlation=0.99; Spearman rank correlation=0.51, FIG. 7C). This showed that whereas individual 5fC sites are transient and dynamic, hotspot regions for 5fC distributions may be relatively stable at a given developmental stage.

Globally, 5hmCpG and 5fCpG sites had similar distributions in genic regions (FIG. 8A). After normalizing to the background CpG density, both 5hmCpG and 5fCpG densities dropped near TSS and remained low at the 5′UTR, but not at the 3′UTR. There was very little difference in the normalized modification levels between exons and introns, although the absolute 5hmCpG and 5fCpG densities were higher in exons than in introns, which is consistent with previous reports (Song, et al., 2013; Yu, et al., 2012). Within exon and intron regions, both 5hmCpG and 5fCpG appeared to gradually increase from the 5′ end to the 3′ end. Our results for the 5hmCpG distribution in genic regions were consistent with previous observations, which showed that the 5hmCpG profile generally follows the 5mCG profile (Sun, et al., (2013)). Here we further demonstrated that the 5fCpG distribution also resembled the distribution of its precursor 5hmCpG, thus indicating that 5hmCs and 5fCs in genic regions are largely shaped by their precursors' availability.

Intriguingly, 5hmCH and 5FCH showed distinct profiles in genic regions from those of 5hmCpG and 5fCpG (FIG. 8B). Normalized 5hmCH and 5FCH levels were elevated in coding regions in comparison to non-coding regions. In contrast to 5hmCpG and 5fCpG profiles, 5hmCH and 5FCH were not depleted near TSS. In addition, 5hmCH and 5FCH gradually decreased towards transcription termination sites (TTS). It has been reported that the 5mCHH density was 15˜20% higher in exons than in introns in human embryonic stem cells (Lister, et al. (2009)). Therefore, the observed distribution of 5hmCH and 5FCH modification in different genic regions might be attributable to 5mC availability.

Although the overall distributions of 5hmC and 5fC were similar in most genic regions, they vary at many protein-DNA interacting sites. With the single-base resolution data we generated for 5hmC and 5fC, it is now possible to evaluate the distribution of 5hmC and 5fC at the protein-DNA binding sites, whose consensus sequences are usually short (<20 bp).

TET protein is responsible for the oxidation from 5mC to 5hmC, 5fC and 5caC successively. Consistent with previous reports (Song, et al., 2013; Williams, et al., Nature, 473:343-348 (2011); Wu, et al., 2011), the absolute level of both 5hmC and 5fC was elevated at TET1-binding sites (FIG. 9A). However, after normalizing 5hmC and 5fC densities to background CpG and CH densities, the elevation became less significant. In contrast, 5hmCH, 5fCpG and 5fC pH were still enriched at TET1 binding sites.

The distributions of 5hmC and 5fC at the binding sites of 13 transcription factors (TFs) (Nanog, Oct4, STAT3, Smad1, Sox2, Zfx, c-Myc, n-Myc, Klf4, Esrrb, Tcfcp2I1, E2f1, and CTCF) and 2 transcription regulators (p300 and Suz12) that are known to play important roles in the ESCs (Chen, et al., (2008)) were analyzed. The results for CTCF, Nanog, and Tcfcp2I1 are shown in FIGS. 9B, 9D and 9E.

As the main insulator-binding protein in vertebrates, CCCTC-binding factor (CTCF) plays an important role in promoting and mediating long-range enhancer-promoter interactions and in establishing functional domains of gene expression (Ong, et al., Nat. Rev. Genet, 15, 234-246 (2014)). Here a symmetrical, regularly-spaced oscillating distribution of 5hmC and 5fC in the CTCF-bound regions, was found coincident with the local nucleosome array structure (Cuddapah, et al., Genome Research, 19:24-32 (2009); Fu, et al., P/os Genet, 4, e1000138 (2008)); both 5hmC and 5fC level peaked at ˜150 bp intervals which overlaid with the length of the linker DNAs between nucleosomes (FIG. 9B) suggesting that local chromatin structure may affect accessibility to the TET enzymes and thus shape the distribution of 5mC oxidation products. Interestingly, we noted that the central 5hmC peaks were lower than neighboring peaks, whereas the central 5fC peaks were higher than neighboring peaks (FIG. 9B).

Similarly, for the co-activator p300 which is known to interact with various transcription factors and mark highly dense binding loci in the genome, both 5hmCs and 5fCs were abundant at the p300 binding sites. 5fC appeared to be slighted more enriched than 5hmC at P300 binding sites (FIG. 9C). Furthermore, 5hmCH and 5fCH were more enriched than 5hmCpG and 5fCpG, suggesting more active demethylation activities in non-CpG context. In human ES cells, the methylation level in p300 binding sites was more depleted in non-CpG context than in CpG context (Lister, et al., (2009)). These data provided supporting evidences for the role of TET1 activity in the change of epigenetics landscape allowing fine tuning of transcriptional regulation.

Distinct 5hmC and 5fC profiles were found for 13 representative transcription factors or regulators. It is anticipated that embodiments of the method can be applied to any site of interest on the genome. Based on the modification profiles of these 13 sites, they were divided into three groups. The first group, including Nanog, Oct4, Sox2, Klf4 and Smad1, displayed depleted 5hmCpG levels but enriched 5fCpG, which might be indicative of constant active demethylation in these binding sites (FIG. 9D). The second group, comprising of Tcfcp2I1 and Esrrb, showed enriched 5hmC and 5fC in both CpG and CH context (FIG. 9E). The third group contained six different transcription factors or regulators: c-Myc, n-Myc, E2f1, Zfx, Stat3 and Suz12. This group appeared to have elevated absolute 5hmC and 5fC levels in both CpG and CH context at the binding sites; but when normalized to CpG density, the enrichment at these sites became insignificant or even disappeared, while in contrast, CH sites still retained higher modification levels relative to the flanking regions (FIG. 9F). While not wishing to be limited to a hypothesis, it might be concluded that regulatory elements have a more variable DNA modification profile than genic regions.

The distribution of modified nucleotides, 5hmC and 5fC were examined at chromatin modification sites. For example, both 5hmC and 5fC were depleted at H3K4me3 chromatin modification sites (FIG. 10A), a hallmark of actively transcribed protein-coding promoters in eukaryotes (Barski, et al., Cell, 129:823-837 (2007); Mikkelsen, et al., 2007). In contrast, 5hmC and 5fC were enriched at repressive chromatin loci marked by H3K27me3 (FIG. 10B). We also examined the correlation between enhancers and 5hmC or 5fC distribution by mapping 5hmC and 5fC reads to chromatin regions that are marked by different combinations of two histone modifications H3K4me1 and H3K27Ac (Creyghton, et al., 2010). While 5hmCs and 5fCs were enriched at both active (H3K4me1 with H3K27Ac) and poised (H3K4me1 without H3K27Ac) enhancers (FIGS. 10C and 10D), the enrichment was stronger at the poised enhancers. These results provide additional support to a close correlation between DNA modification and transcription regulation.

1. A preparation comprising a PvuRts1I-family bacterial restriction endonuclease and a eukaryotic DNA having at least one 5-hydroxymethylcytosine (5hmC).

2. A preparation according to claim 1 wherein the molar ratio of the bacterial restriction endonuclease to 5-hydroxymethylcytosine (5hmC) in the eukaryotic DNA is at least 0.5:1.

3. A preparation according to claim 1, further comprising a glucosyltransferase and a glucosyltransferase substrate that comprises a chemo-selective group.

4. A preparation according to claim 1, further comprising, an adapter having at least a two nucleotide 3′ overhang of random sequence and a 5′ phosphate for ligating to the eukaryotic DNA.

5. A method for detecting 5-hydroxymethylcytosine (5hmC) in a sample, comprising:
(a) digesting eukaryotic genomic DNA comprising 5hmC using a preparation according to claim 1, to form a DNA having a first end, wherein the first end has a single strand overhang; the eukaryotic genomic DNA being randomly fragmented (i) prior to restriction endonuclease digestion, or (ii) after restriction endonuclease digestion;
(b) ligating an adapter to the first end; and
(c) detecting the presence and the position of 5hmC in the eukaryotic genomic DNA using DNA sequences determined for the adaptor ligated DNA.

6. The method of claim 5, further comprising selectively adding a chemoselective group to the 5-hydroxymethylcytosine (5hmC) in the DNA prior to (c).

7. The method according to claim 6, wherein selectively adding the chemoselective group comprises reacting the DNA with a glucosyltransferase and a glucosyltransferase substrate.

8. The method according to claim 6, further comprising reacting the added chemoselective group with a capture molecule that comprises an affinity moiety and optionally a cleavable linker; capturing the DNA that comprise the affinity moiety on a matrix; and releasing the captured DNA from the matrix.

9. The method according to claim 8, wherein releasing the captured DNA comprises cleaving the cleavable linker.

10. The method according to claim 8, wherein the cleavable linker is a disulfide bond, and the cleaving comprises reducing the disulfide bond.

11. The method according to claim 8, wherein the affinity moiety is a biotin moiety.

12. A method according to claim 6, wherein selectively adding the chemoselective group to 5-hydroxymethylcytosine (5hmC) further comprises increasing the reaction temperature to 37° C.

13. A method according to claim 5, wherein (a) further comprises, digesting the eukaryotic genomic DNA with the restriction endonuclease at a temperature below 37° C.

14. A method according to claim 5, further comprising: combining the PvuRts1I-family restriction endonuclease, a glucosyltransferase, a glucosyltransferase substrate and the eukaryotic genomic DNA in a single reaction vessel.

15. A method according to claim 5, further comprising removing restriction endonuclease activity prior to ligating the adapter.

16. A method according to claim 13, further comprising heat inactivating the restriction endonuclease.

17. The method according to claim 5 wherein an amount of the restriction endonuclease corresponds to a molar ratio of the restriction endonuclease to total 5-hydroxymethylcytosine (5hmC) in the eukaryotic DNA of at least 0.5:1.

18. The method according to claim 1, wherein the first end is characterized by a 3′ two base overhang on a strand of the eukaryotic genomic DNA having a 5-hydroxymethylcytosine (5hmC).

19. The method according to claim 18, wherein the overhang is a two nucleotide overhang of random sequence.

20. The method according to claim 1, further comprising randomly fragmenting the digestion products into DNA fragments having a size of less than 500 bases.

21. The method according to claim 5, wherein the eukaryotic DNA has a second end, the method comprising adding a second adapter to the second end for amplifying the eukaryotic DNA between the adapters at the first end and the second end.

22. The method according to claim 5, further comprising (d) annotating a cytosine as being a 5-hydroxymethylcytosine (5hmC) or 5-formylcytosine (5fC) in the eukaryotic genomic DNA.

23. The method according to claim 5, further comprising treating the eukaryotic DNA with NaBH₄ prior to (a).

24. A kit comprising a PvuRts1I-family restriction endonuclease, a glucosyltransferase and a glucosyltransferase substrate that comprises a chemo-selective group and a buffer and instructions for use at an initial temperature of room temperature followed by an incubation at 37° C.


SEQUENCE LISTING (12 sequences)

SEQ ID NO: 1 - DNA, 53 bp, Artificial Sequence
  Synthetic construct
ggttggactcaagacgatattaccggataaggcgcaattagattacttaacct

SEQ ID NO: 2 - DNA, 53 bp, Artificial Sequence; identical to SEQ ID NO: 1
  Synthetic construct

SEQ ID NO: 3 - DNA, 53 bp, Artificial Sequence
  Synthetic construct
aggttaagtaattaattgcgccttatccggtaagtatcgtcttgagtccaacc

SEQ ID NO: 4 - DNA, 53 bp, Artificial Sequence; identical to SEQ ID NO: 3
  Synthetic construct

SEQ ID NO: 5 - DNA, 54 bp, Artificial Sequence
  Synthetic construct
aggttaagtaatctaattgcgccttatccggtaagtatcgtcttgagtccaacc

SEQ ID NO: 6 - DNA, 53 bp, Artificial Sequence
  Synthetic construct
ggttggactcaagacgatattaccggataaggcgcaattatattacttaacct

SEQ ID NO: 7 - DNA, 54 bp, Artificial Sequence
  Synthetic construct
aggttaagtaatataattgcgccttatccggtaagtatcgtcttgagtccaacc

SEQ ID NO: 8 - DNA, 54 bp, Artificial Sequence
  Synthetic construct
ggttggactcaagacgatacttaccggataaggcgcaattagattacttaacct

SEQ ID NO: 9 - DNA, 35 bp, Artificial Sequence
  Synthetic construct
acactctttccctacacgacgctcttccgatctnn

SEQ ID NO: 10 - DNA, 33 bp, Artificial Sequence
  Synthetic construct
agatcggaagagcgtcgtgtagggaaagagtgt

SEQ ID NO: 11 - DNA, 31 bp, Artificial Sequence
  Synthetic construct
gatcggaagagcacacgtctgaactccagtc

SEQ ID NO: 12 - DNA, 32 bp, Artificial Sequence
  Synthetic construct
gactggagttcagacgtgtgctcttccgatct